(19) United States
(12) Patent Application Publication
(10) Pub. No.: US 2026/0306112 A1
(43) Pub. Date: Oct. 8, 2026

(54) RIGHT ANGLE DRILL, INSTRUMENTATION, SYSTEM AND METHODS OF USE AND ASSEMBLY

(71) Applicant: Paragon 28, Inc., Englewood, CO (US)

(72) Inventors: Zachary Ryan WOODS, Centennial, CO (US); Dylan PAPROSKI, Littleton, CO (US); Darby MACLEISH, Denver, CO (US); Caitlin REUTER, Littleton, CO (US); Daniel J. Lee, Denver, CO (US); Sven BLACHE, Wellendingen (DE); Marc MUNDHENKE, Rielasingen-Worblingen (DE); Duncan MELTON, Centennial, CO (US)

(73) Assignee: Paragon 28, Inc., Englewood, CO (US)

(21) Appl. No.: 19/683,560

(22) Filed: May 20, 2026

Related U.S. Application Data
(63) Continuation of application No. PCT/US2024/056688, filed on Nov. 20, 2024.
(60) Provisional application No. 63/601,047, filed on Nov. 20, 2023.

(51) Int. Cl. A61B 17/16; A61B 17/00; A61B 17/17

(52) U.S. Cl. CPC A61B 17/1624; A61B 17/1775; A61B 2017/00477

(57) ABSTRACT

Right angle drills, instruments, systems and methods for arthroplasty procedures of the ankle joint. The instrument including a coupling portion, an alignment assembly coupled to the coupling portion, and a driving member coupled to the alignment assembly, wherein the drill bit is positioned perpendicular to a housing of the driving members. The instrument having a drill bit to drill a central opening in a tibial resection.

### CROSS REFERENCE TO RELATED APPLICATIONS

This application is a continuation of International Application No. PCT/US 2024/056688 filed Nov. 20, 2024 and entitled Right Angle Drill, Instrumentation, System and Methods of Use and Assembly, which claims priority benefit under 35 U.S.C. § 119(e) of U.S. Provisional Application No. 63/601,047 filed Nov. 20, 2023, which is incorporated by reference in its entirety.

### TECHNICAL FIELD

The present disclosure relates generally to general, podiatric, and orthopaedic surgery related to joint deformities and injuries. More specifically, but not exclusively, the present disclosure relates to right angle drills, instruments, systems and methods for arthroplasty procedures of the ankle joint.

### BACKGROUND OF THE INVENTION

Many currently available instruments, systems, and surgical methods for procedures involving the foot and/or ankle do not completely address the needs of patients. Additionally, many currently available instruments, systems, and surgical methods for incorporation in procedures involving the foot and/or ankle, for example, ankle arthroplasty procedures, fail to account for properties of joint anatomy and associated mechanical and kinematic movement patterns and/or capabilities.

Thus, it is an object of the present disclosure to overcome one or more of the above-described drawbacks and/or disadvantages of the currently available systems.

### SUMMARY OF THE INVENTION

The present disclosure is directed toward instruments, systems and methods for procedures involving the foot and/or ankle.

In one aspect of the present disclosure provided herein, is an instrument. The instrument includes a coupling portion, an alignment assembly coupled to the coupling portion, and a driving member coupled to the alignment assembly, wherein a drill bit is positioned perpendicular to a housing of the driving member.

In another aspect of the present disclosure provided herein is a surgical method for ankle arthroplasty procedures.

These and other objects, features and advantages of this disclosure will become apparent from the following detailed description of the various aspects of the disclosure taken in conjunction with the accompanying drawings.

### BRIEF DESCRIPTION OF THE DRAWINGS

The accompanying drawings, which are incorporated in and constitute a part of the specification, illustrate embodiments of the disclosure and together with the detailed description herein, serve to explain the principles of the disclosure. It is emphasized that, in accordance with the standard practice in the industry, various features are not drawn to scale. In fact, the dimensions of the various features may be arbitrarily increased or reduced for clarity of discussion. The drawings are only for purposes of illustrating preferred embodiments and are not to be construed as limiting the disclosure.

FIG. 1 is a first perspective view of an instrument system including a trial assembly, in accordance with an aspect of the present disclosure;

FIG. 2 is a second perspective view of the instrument system of FIG. 1, in accordance with an aspect of the present disclosure;

FIG. 3 is a first perspective view of the instrument of FIG. 1 without the trial assembly, in accordance with an aspect of the present disclosure;

FIG. 4 is a second perspective view of the instrument of FIG. 3, in accordance with an aspect of the present disclosure;

FIG. 5 is a first side view of the instrument of FIG. 3, in accordance with an aspect of the present disclosure;

FIG. 6 is a second side view of the instrument of FIG. 3, in accordance with an aspect of the present disclosure;

FIG. 7 is a top view of the instrument of FIG. 3, in accordance with an aspect of the present disclosure;

FIG. 8 is a bottom view of the instrument of FIG. 3, in accordance with an aspect of the present disclosure;

FIG. 9 is a first end view of the instrument of FIG. 3, in accordance with an aspect of the present disclosure;

FIG. 10 is a second end view of the instrument of FIG. 3, in accordance with an aspect of the present disclosure;

FIG. 11 is an exploded, first perspective view of the instrument of FIG. 3, in accordance with an aspect of the present disclosure;

FIG. 12 is an exploded, second perspective view of the instrument of FIG. 3, in accordance with an aspect of the present disclosure;

FIG. 13 is an exploded, first perspective view of a coupling portion of the instrument of FIG. 3, in accordance with an aspect of the present disclosure;

FIG. 14 is an exploded, second perspective view of the coupling portion of FIG. 13, in accordance with an aspect of the present disclosure;

FIG. 15 is an exploded, first side view of the coupling portion of FIG. 13, in accordance with an aspect of the present disclosure;

FIG. 16 is an exploded, second side view of the coupling portion of FIG. 13, in accordance with an aspect of the present disclosure;

FIG. 17 is an exploded, top view of the coupling portion of FIG. 13, in accordance with an aspect of the present disclosure;

FIG. 18 is an exploded, bottom view of the coupling portion of FIG. 13, in accordance with an aspect of the present disclosure;

FIG. 19 is an exploded, first end view of the coupling portion of FIG. 13, in accordance with an aspect of the present disclosure;

FIG. 20 is an exploded, second end view of the coupling portion of FIG. 13, in accordance with an aspect of the present disclosure;

FIG. 21 is an exploded, first perspective view of an alignment assembly of the instrument of FIG. 3, in accordance with an aspect of the present disclosure;

FIG. 22 is an exploded, second perspective view of the alignment assembly of FIG. 21, in accordance with an aspect of the present disclosure;

FIG. 23 is an exploded, first side view of the alignment assembly of FIG. 21, in accordance with an aspect of the present disclosure;

FIG. 24 is an exploded, second side view of alignment assembly of FIG. 21, in accordance with an aspect of the present disclosure;

FIG. 25 is an exploded, top view of the alignment assembly of FIG. 21, in accordance with an aspect of the present disclosure;

FIG. 26 is an exploded, bottom view of the alignment assembly of FIG. 21, in accordance with an aspect of the present disclosure;

FIG. 27 is an exploded, first end view of the alignment assembly of FIG. 21, in accordance with an aspect of the present disclosure;

FIG. 28 is an exploded, second end view of the alignment assembly of FIG. 21, in accordance with an aspect of the present disclosure;

FIG. 29 is an exploded, first perspective view of a driving member of the instrument of FIG. 3, in accordance with an aspect of the present disclosure;

FIG. 30 is an exploded, second perspective view of the driving mechanism of FIG. 29, in accordance with an aspect of the present disclosure;

FIG. 31 is an exploded, first side view of the driving mechanism of FIG. 29, in accordance with an aspect of the present disclosure;

FIG. 32 is an exploded, second side view of the driving member of FIG. 29, in accordance with an aspect of the present disclosure;

FIG. 33 is an exploded, top view of the driving member of FIG. 29, in accordance with an aspect of the present disclosure;

FIG. 34 is an exploded, bottom view of the driving member of FIG. 29, in accordance with an aspect of the present disclosure;

FIG. 35 is an exploded, first end view of the driving member of FIG. 29, in accordance with an aspect of the present disclosure;

FIG. 36 is an exploded, second end view of the driving member of FIG. 29, in accordance with an aspect of the present disclosure; and

FIG. 37 is an elevated, front perspective view of a component of an instrument system, in accordance with an aspect of the present disclosure;

FIG. 38 is an elevated, rear perspective view of the component of FIG. 37 of an instrument system, in accordance with the present disclosure;

FIG. 39 is an alternate perspective view of the component of FIG. 37 of an instrument system, in accordance with the present disclosure;

FIG. 40 is a front view of the component of FIG. 37 of an instrument system, in accordance with the present disclosure;

FIG. 41 is a rear view of the component of FIG. 37 of an instrument system, in accordance with the present disclosure;

FIG. 42 is a top view of the component of FIG. 37 of an instrument system, in accordance with the present disclosure;

FIG. 43 is a bottom view of the component of FIG. 37 of an instrument system, in accordance with the present disclosure; and

FIG. 44 is cross-sectional view of the component of FIG. 37 of an instrument system, in accordance with the present disclosure.

### DETAILED DESCRIPTION FOR CARRYING OUT THE INVENTION

Generally stated, disclosed herein are right angle drills, instruments, and systems for arthroplasty procedures, for example, arthroplasty procedures of the ankle joint. Further, methods for arthroplasty procedures of the ankle joint are discussed.

In this detailed description and the following claims, the words proximal, distal, anterior or plantar, posterior or dorsal, medial, lateral, superior, and inferior are defined by their standard usage for indicating a particular part or portion of a bone, instrument, or implant according to the relative disposition of the natural bone or directional terms of reference. For example, “proximal” means the portion of an instrument nearest the torso, while “distal” indicates the portion of the instrument farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure. Further, specifically in regards to the foot, the term “dorsal” refers to the top of the foot and the term “plantar” refers to the bottom of the foot.

Similarly, positions or directions may be used herein with reference to anatomical structures or surfaces. For example, as the current instrumentation, system, and methods are described herein with reference to use with the bones of the ankle, the bones of the foot, ankle and lower leg may be used to describe the surfaces, positions, directions or orientations of the instrumentation, system, and methods. Further, the instrumentation, system, and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the instrumentation, system, and methods, and the aspects, components, features and the like thereof, described and/or illustrated herein may be changed, varied, modified, reconfigured or otherwise altered for use or association with another side of the body for a same or similar purpose without departing from the spirit and scope of the disclosure. For example, the instrumentation, system, and methods, and the aspects, components, features and the like thereof, described herein with respect to the right leg may be mirrored so that they likewise function with the left leg. Further, the instrumentation, system, and methods, and the aspects, components, features and the like thereof, disclosed herein are described with respect to the leg for brevity purposes, but it should be understood that the instrumentation, system, and methods may be used with other bones of the body having similar structures.

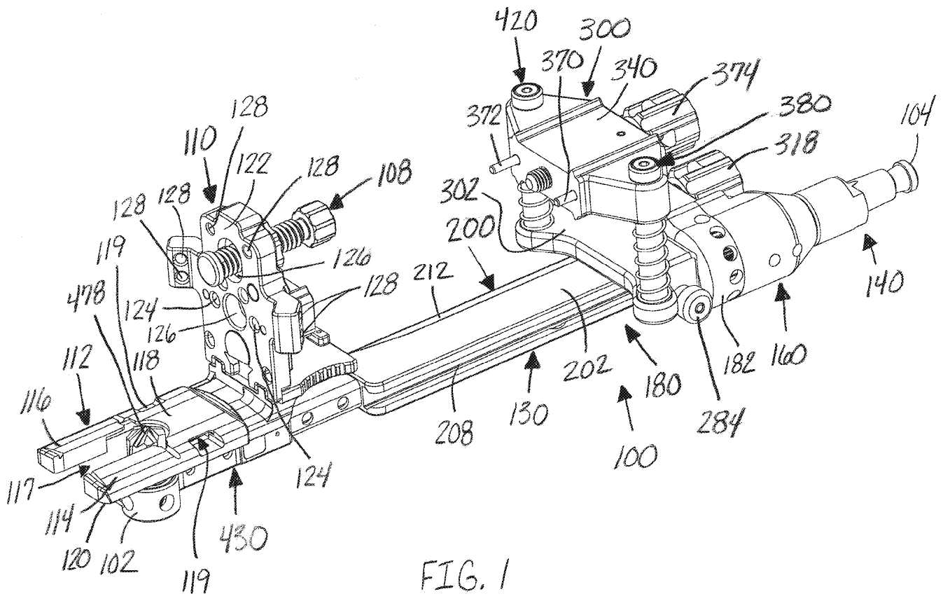

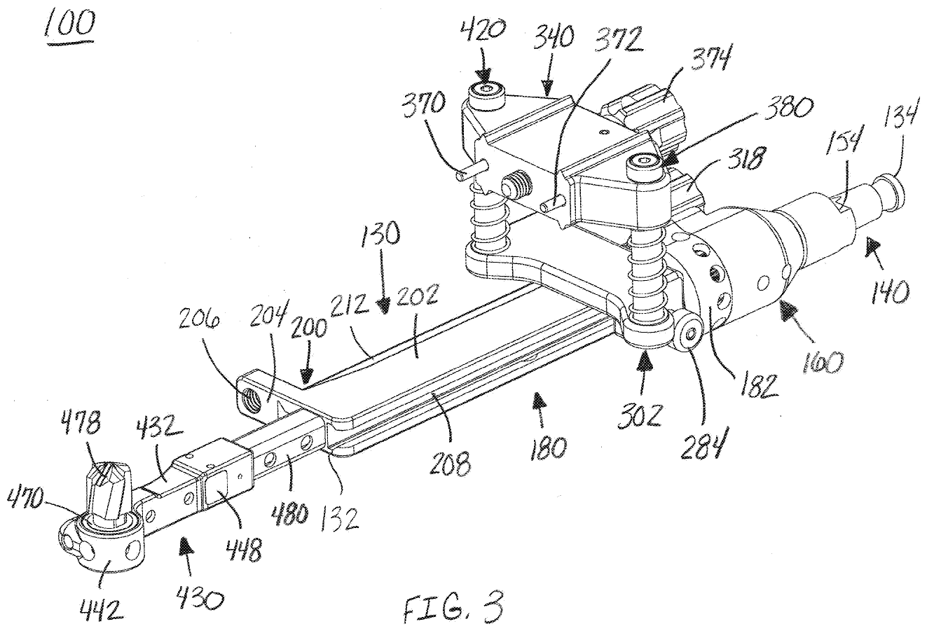

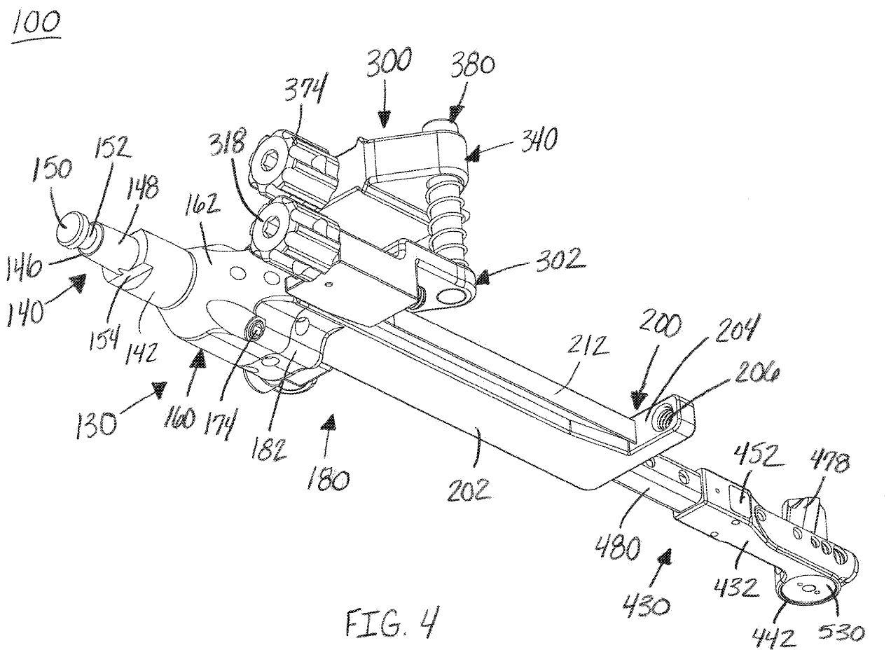

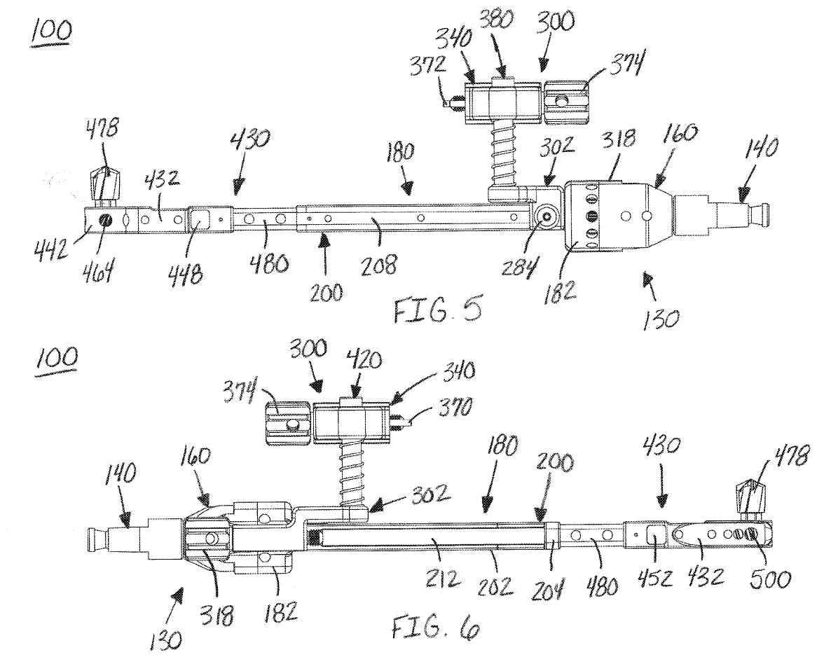

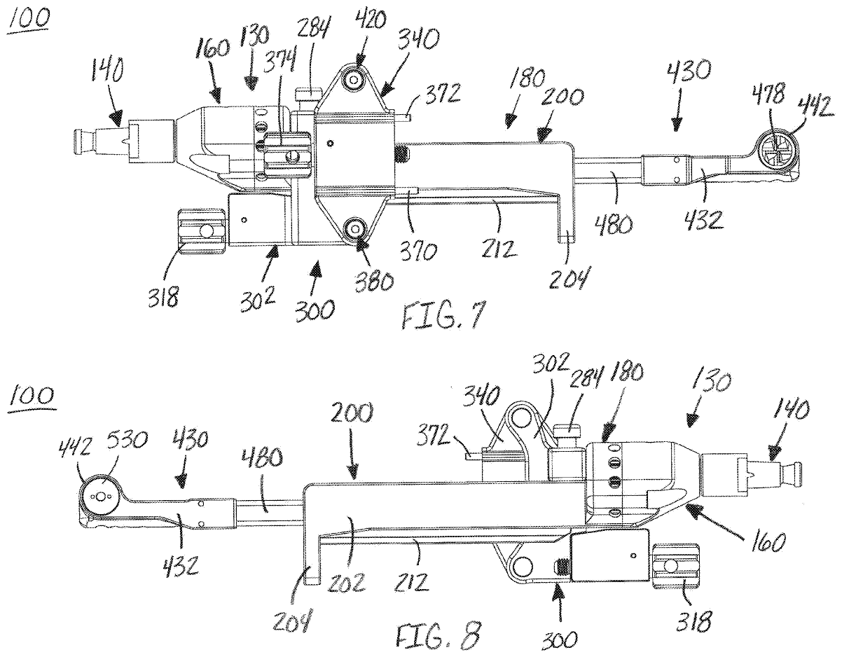

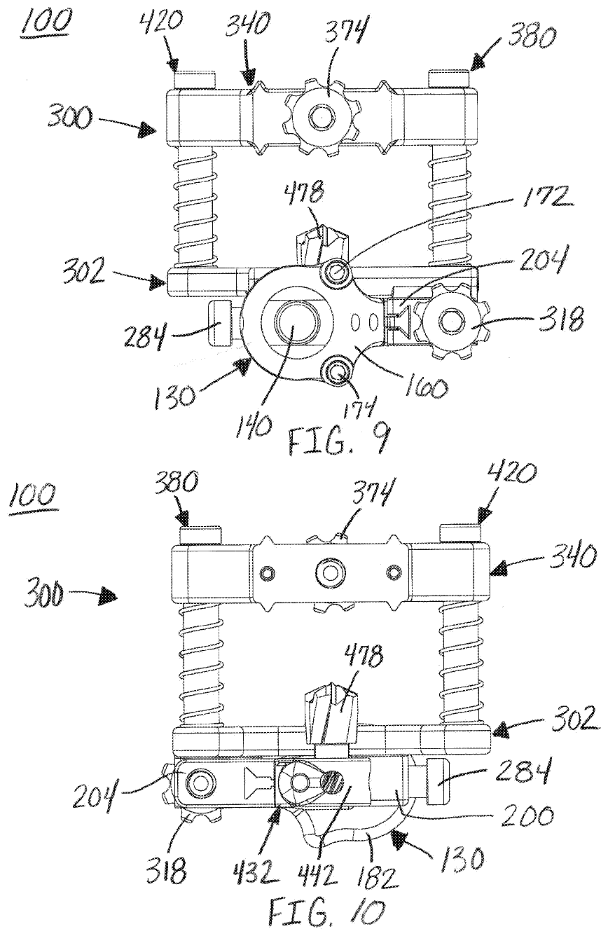

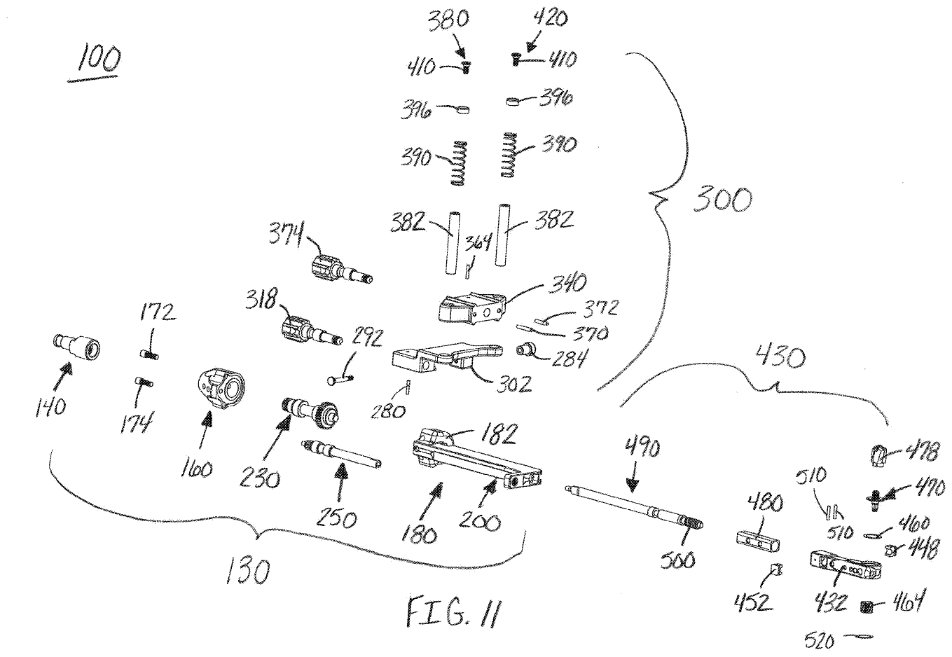

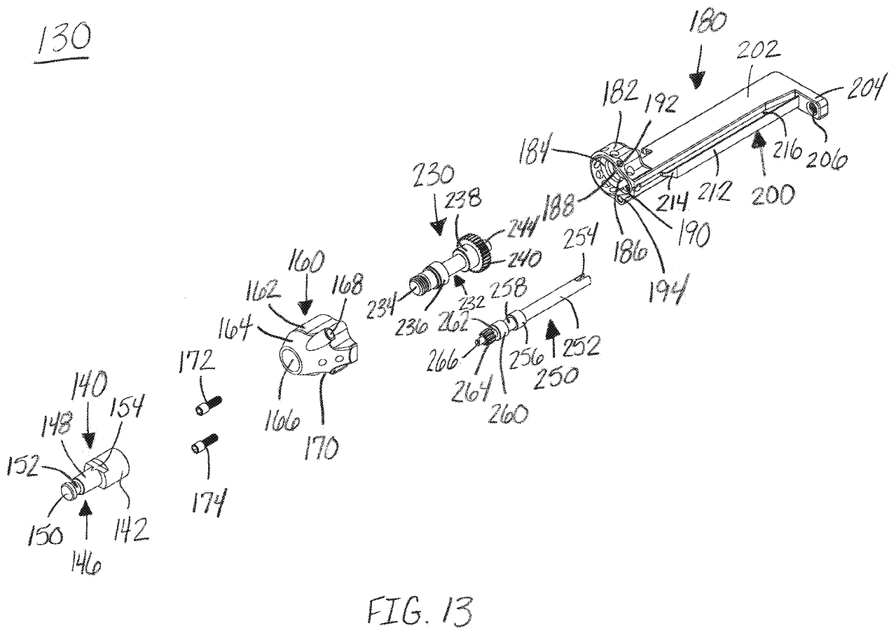

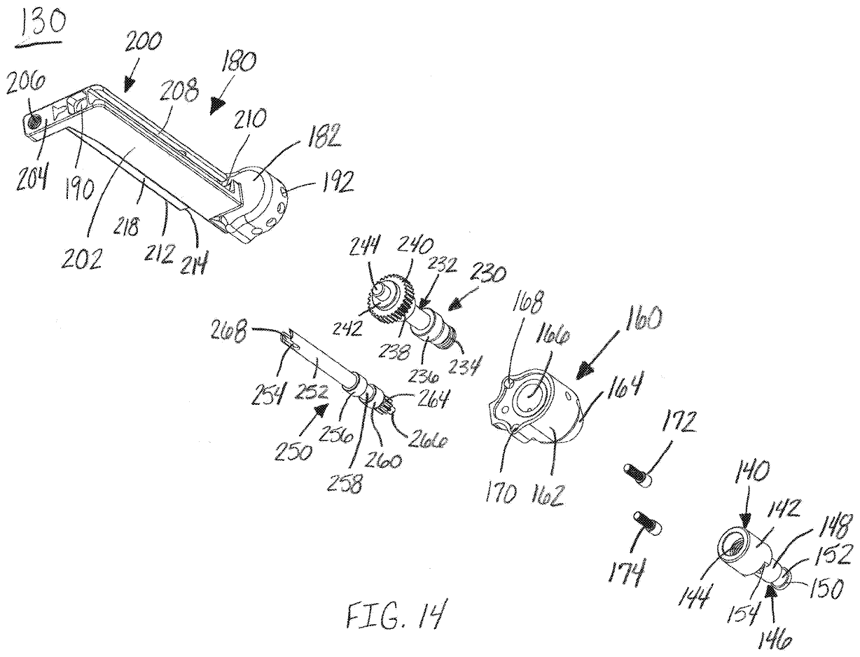

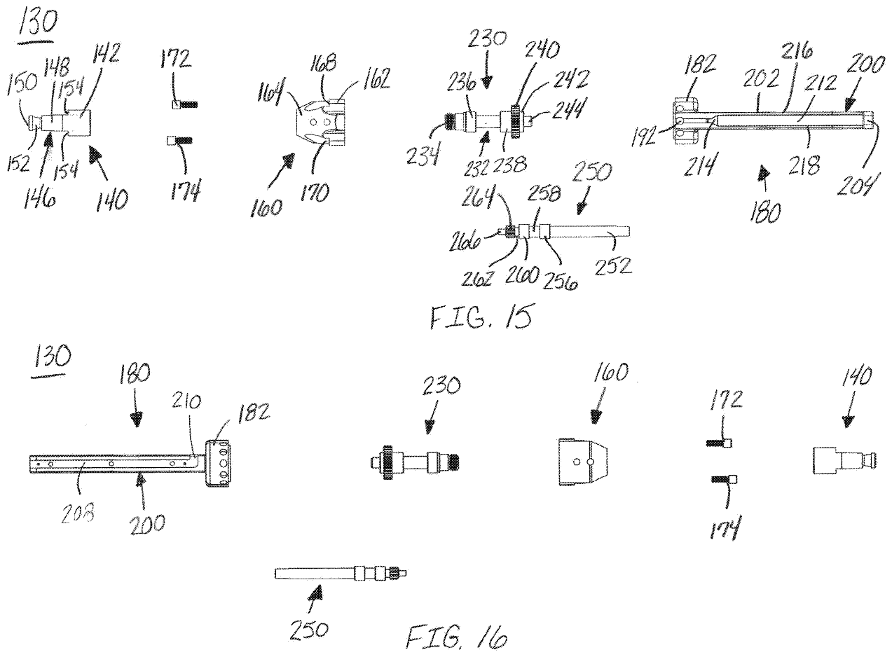

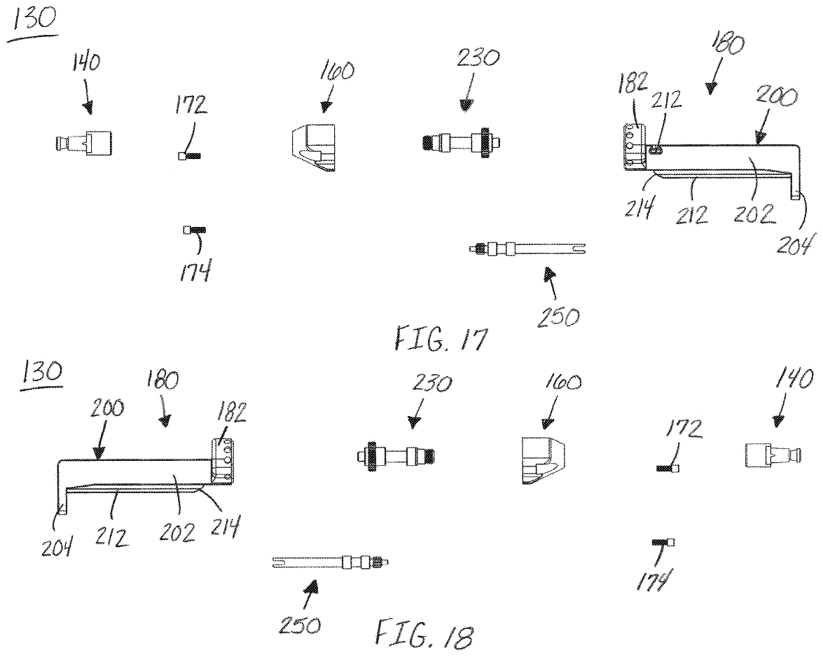

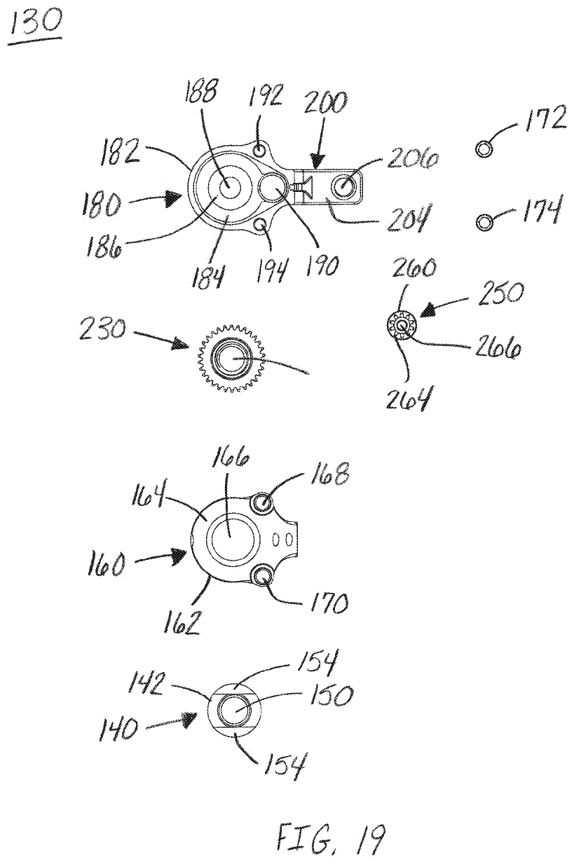

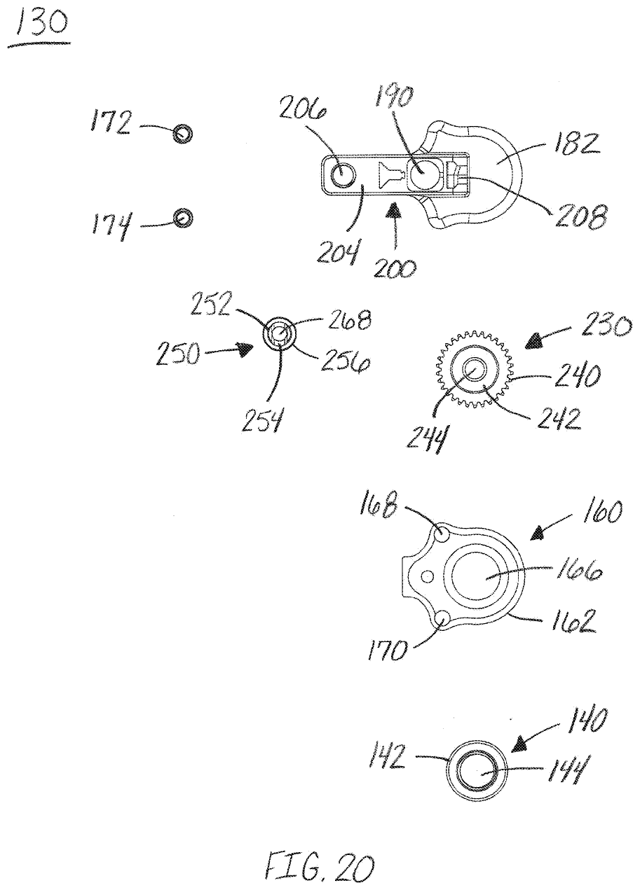

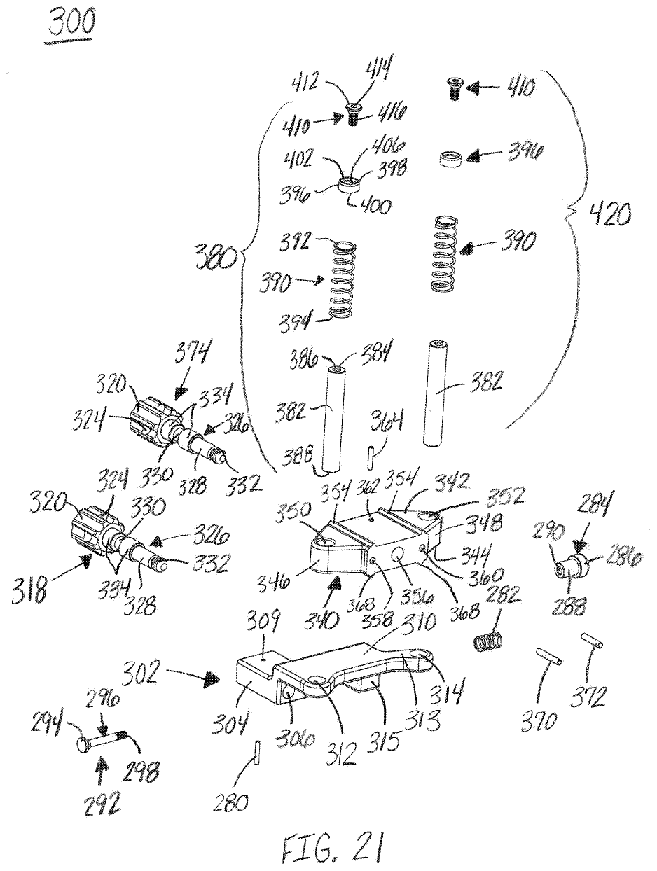

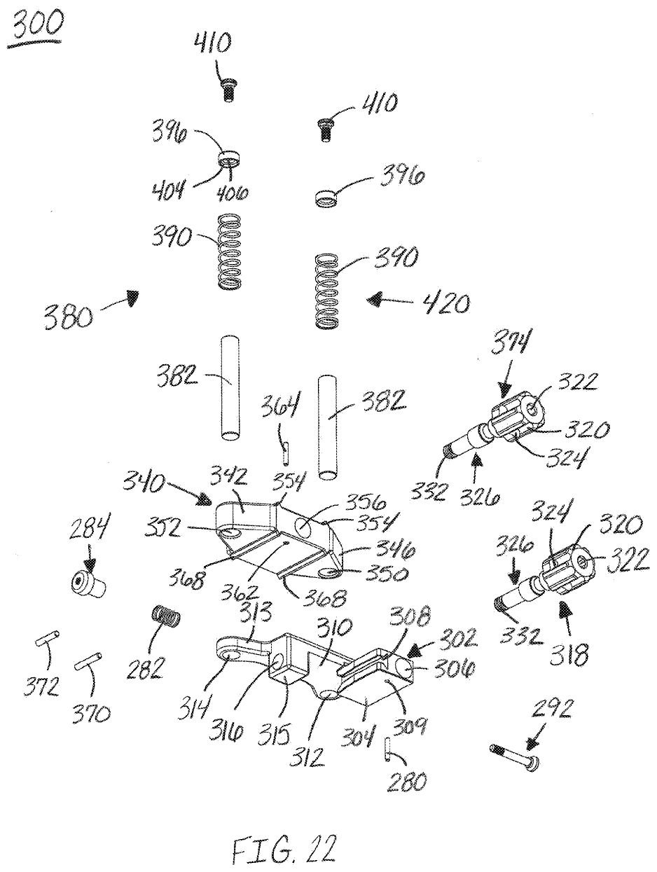

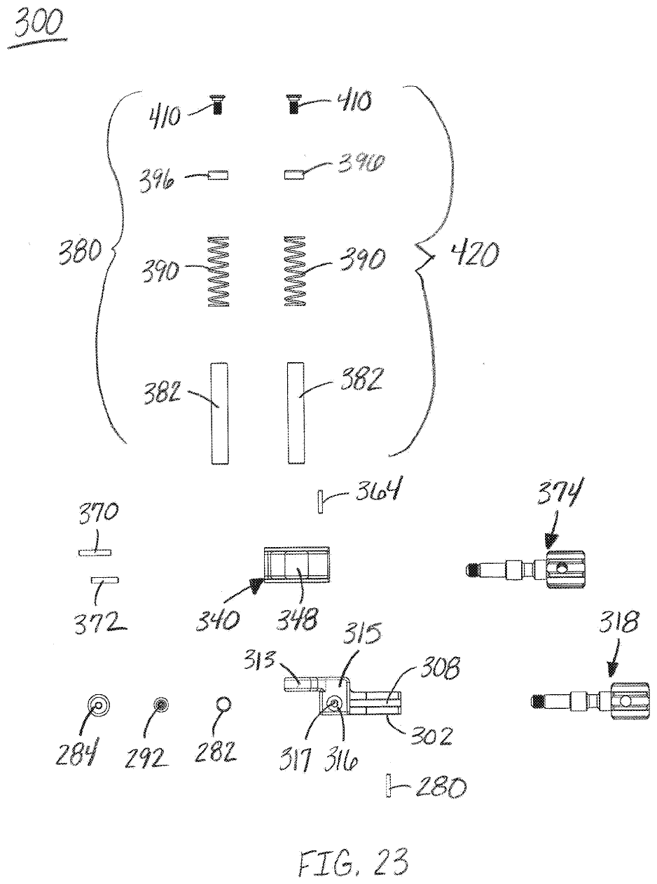

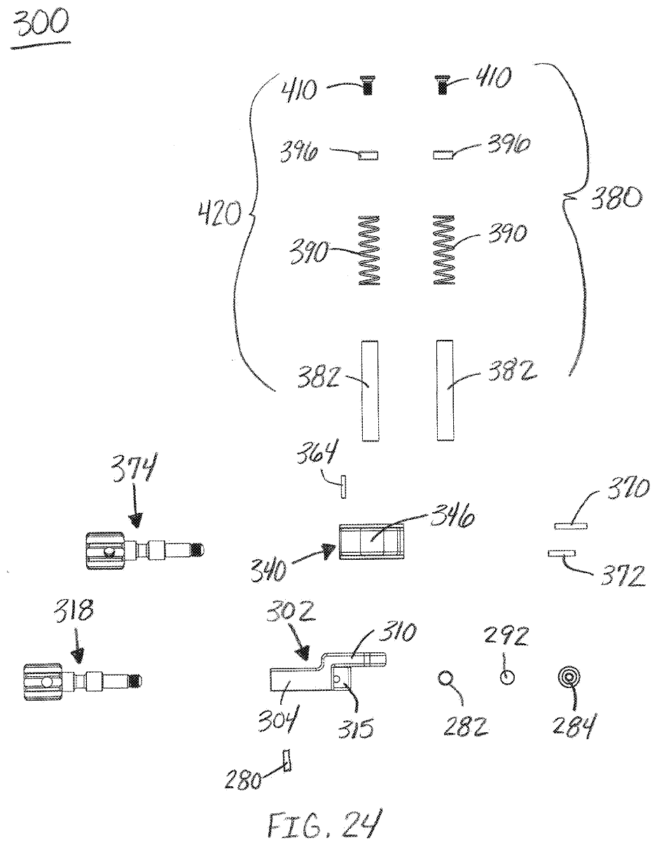

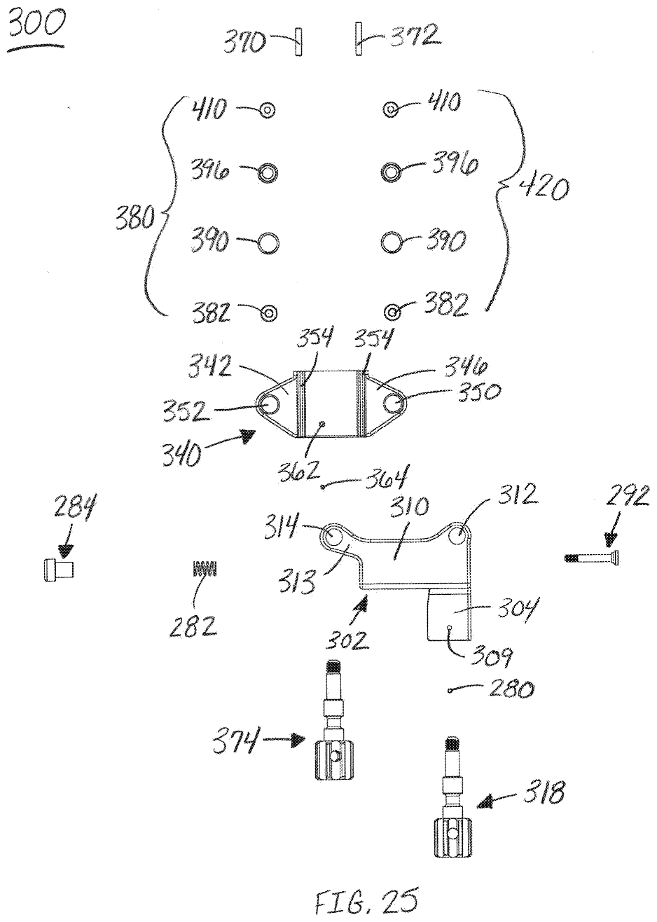

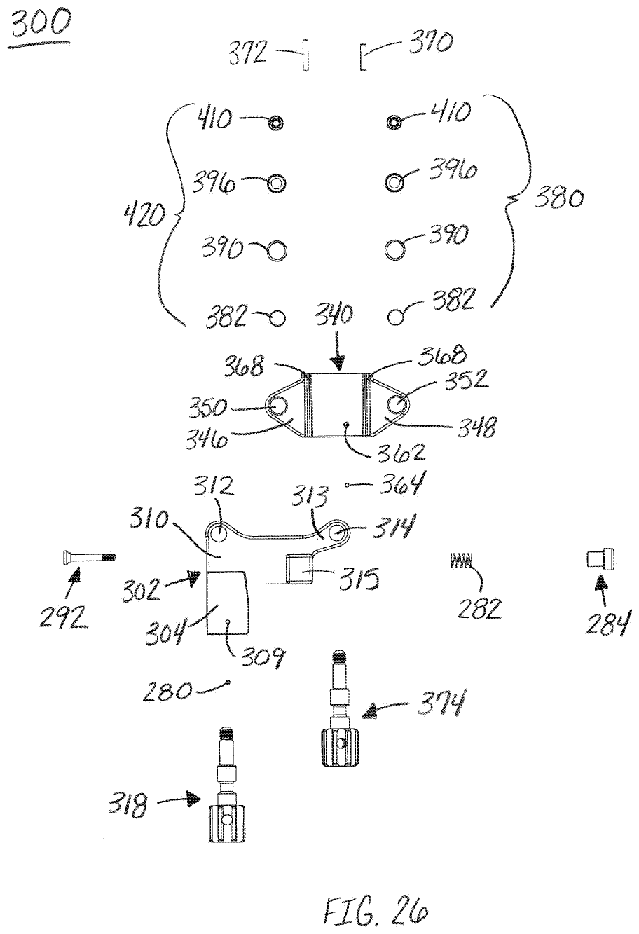

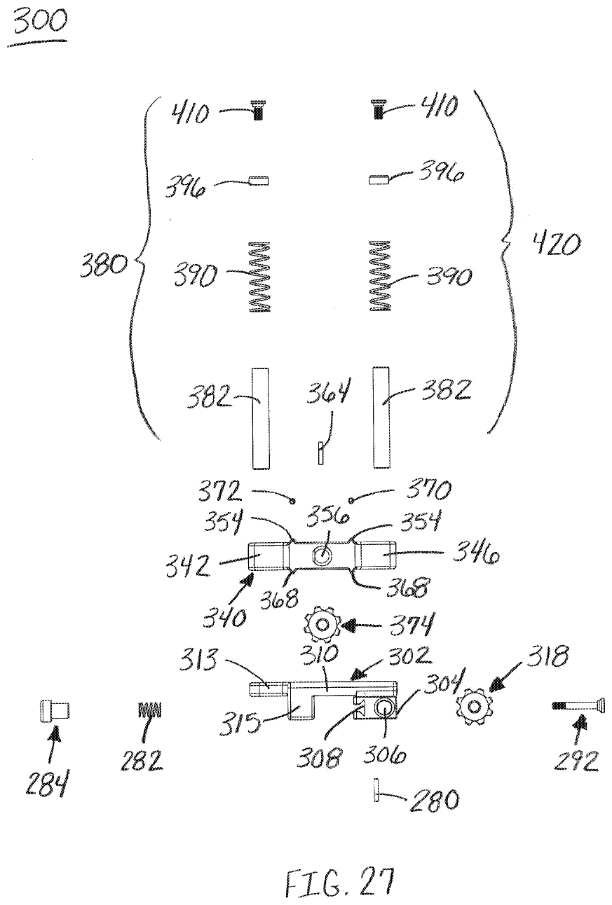

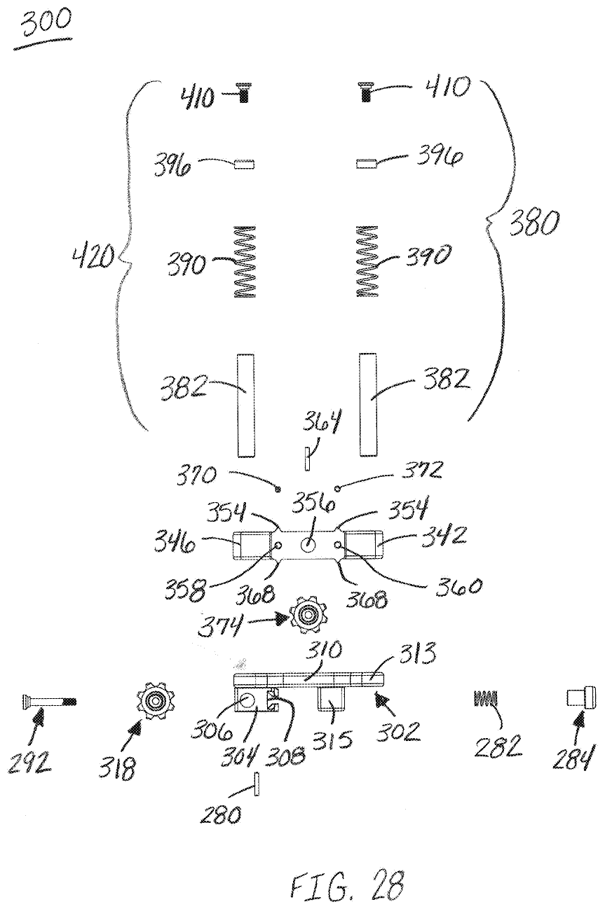

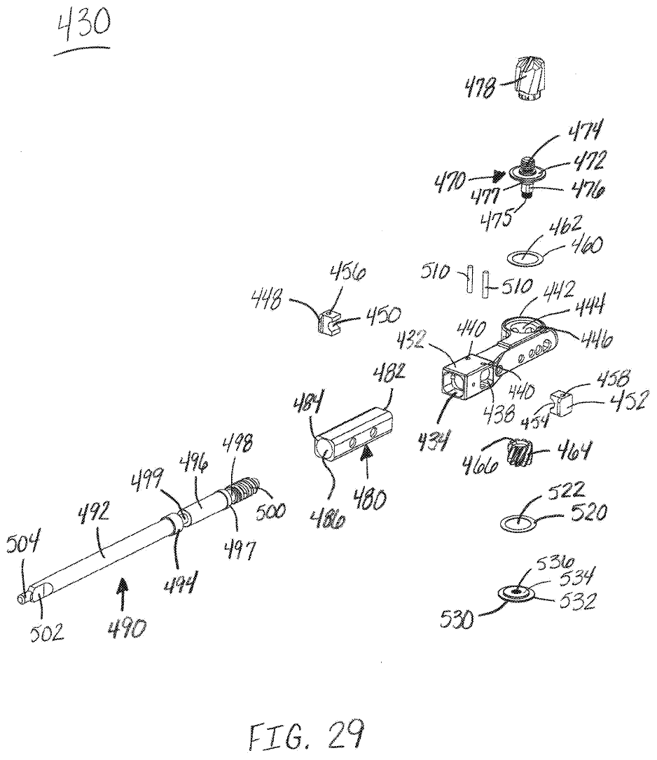

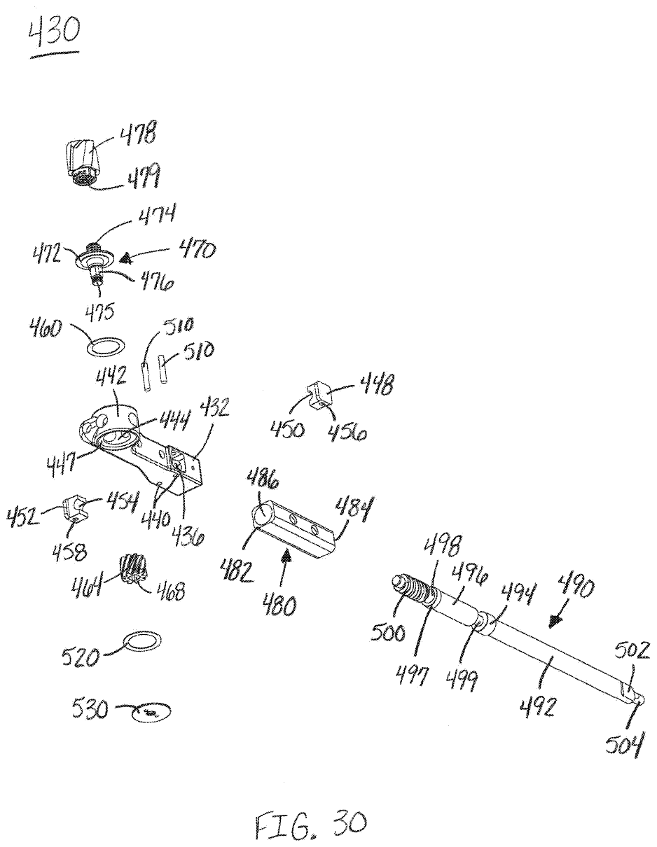

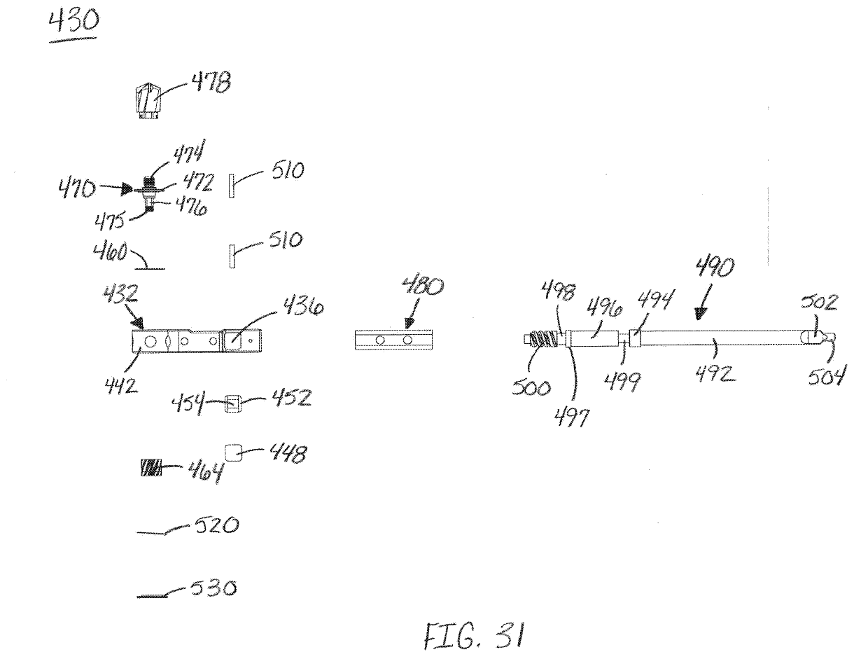

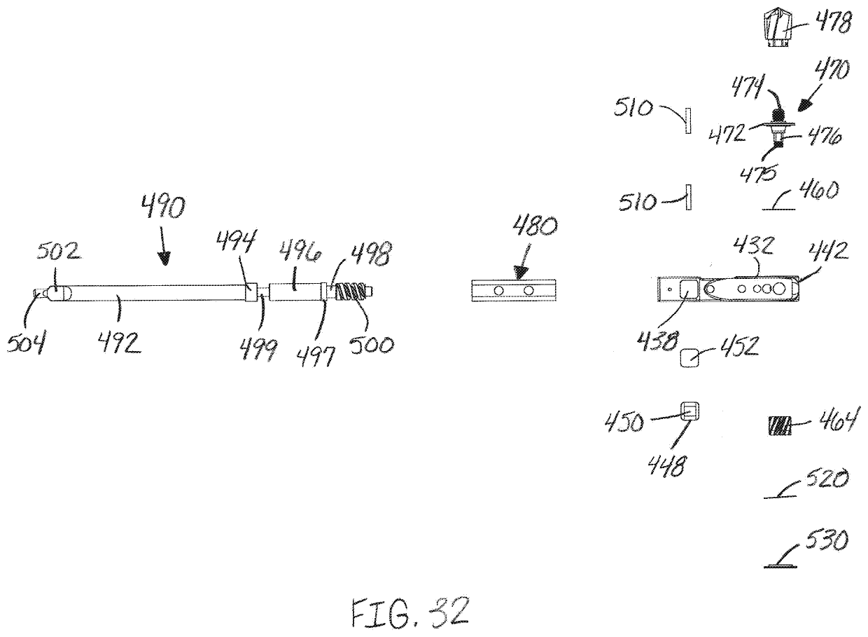

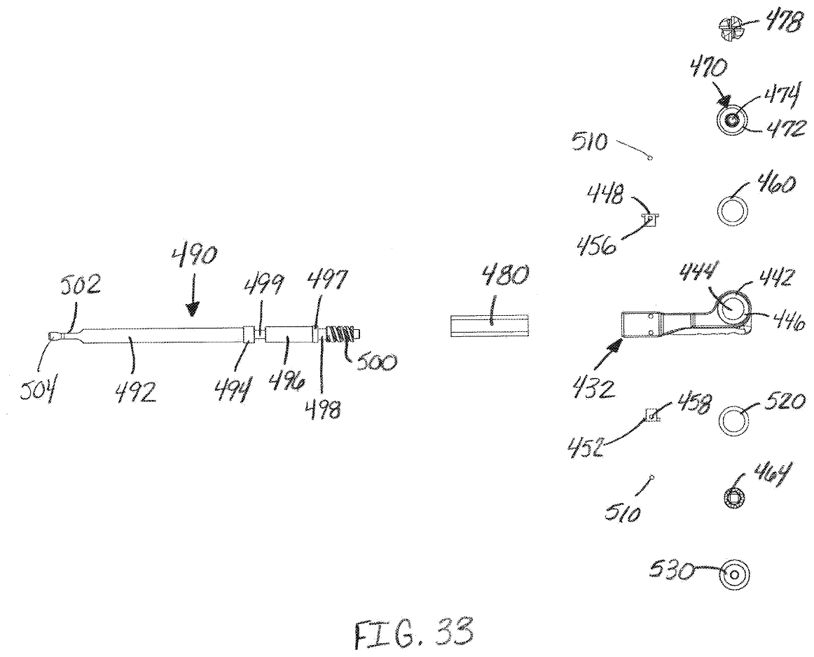

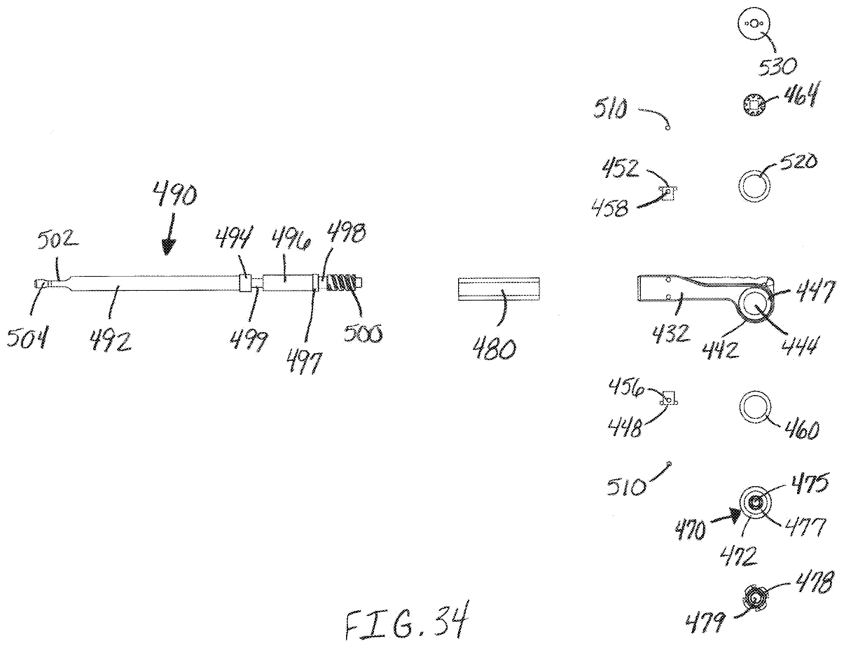

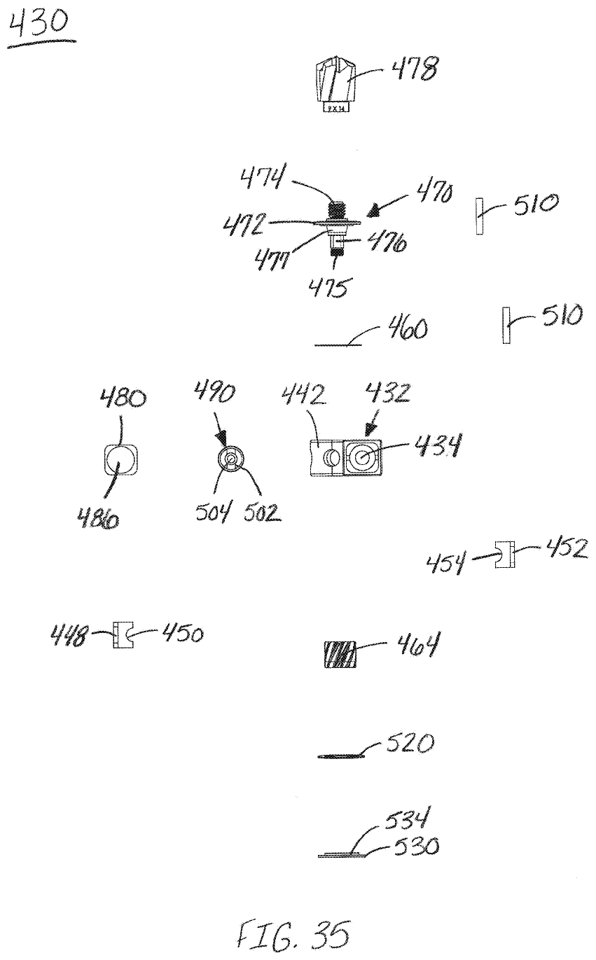

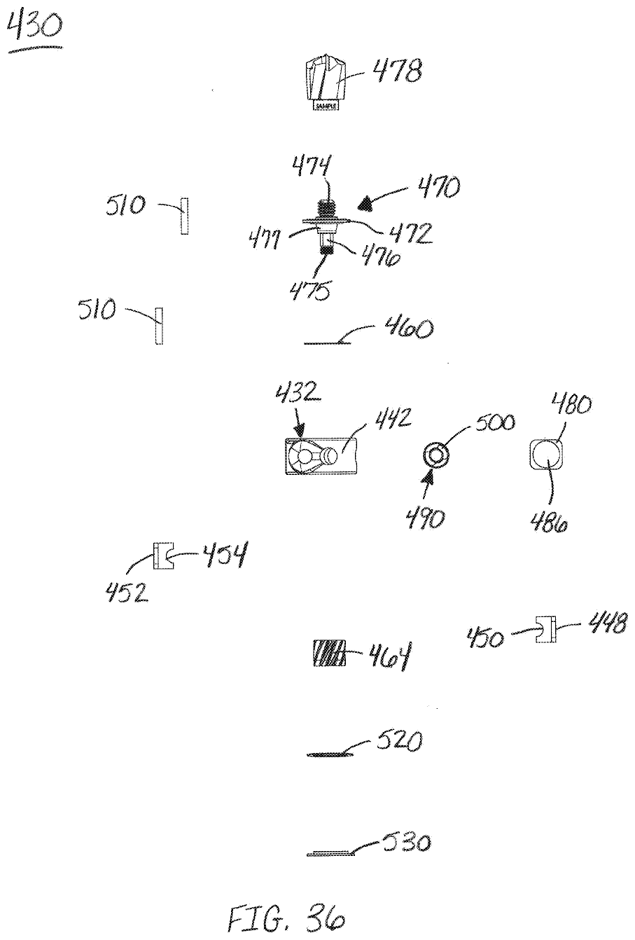

Referring to the drawings, wherein like reference numerals are used to indicate like or analogous components throughout the several views, and with particular reference to FIGS. 1-36, instrumentation, system, and methods of using the instrumentation, system, and methods for arthroplasty procedures of the ankle joint or like joints are shown.

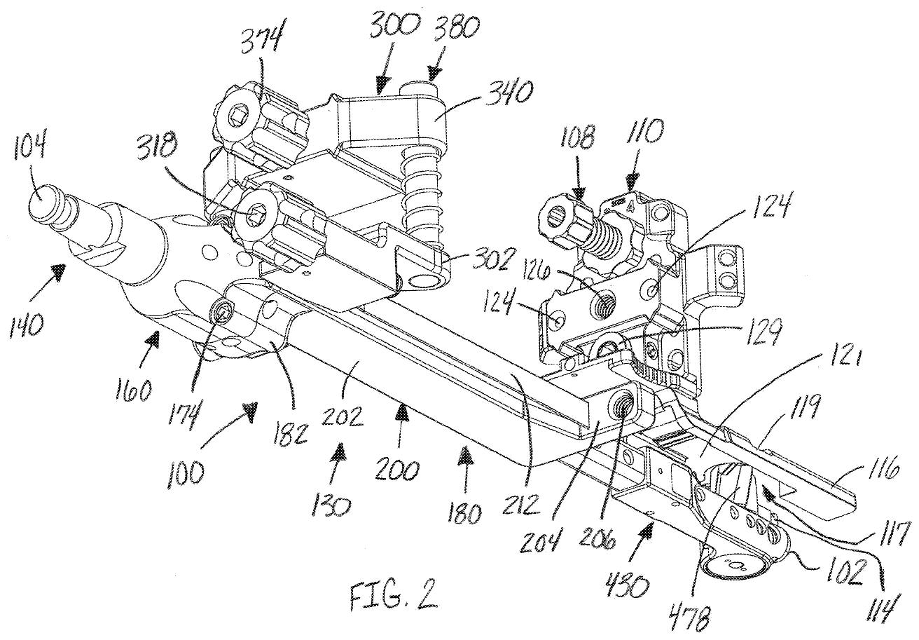

Referring now to FIGS. 1 and 2, an instrument or drill 100 coupled to a tibial cutting guide 110 are shown. The instrument 100 with a central drill bit 478 is shown in FIGS. 1 and 2. The instrument 100 couples to the tibial cutting guide 110. The cutting guide 110 includes a tibial tray and/or tibial trial 112 and a tibial coupling element 122 that is coupled to the tibial tray 112, for example, perpendicularly or at a right angle. The tibial tray 112 includes a first arm 114 and a second arm 116 with a drill channel 117 positioned between the arms 114, 116. The drill channel 117 may extend through the first or top surface 118 to the second or bottom surface 120 between the first arm 114 and the second arm 116. The drill channel 117 may be positioned to extend from a first end toward a midpoint of the tibial tray 112. The drill channel 117 may be, for example, configured or sized and shaped to receive the drill bit 478 of the instrument 100. The tibial coupling element 122 may include at least one attachment opening 124 for aligning the tibial cutting guide 110 to the alignment assembly 300 of the instrument 100. The tibial coupling element 122 further includes at least one securement opening 126 for coupling the tibial cutting guide 110 to the alignment assembly 300 of the instrument 100.

Referring now to FIGS. 1-36, embodiments of an instrument, drill or right angle drill 100 are shown. The instrument 100 includes a drill engagement portion 130, an alignment assembly 300, and a driving member 430. The driving member 430 extends from a first end 102 toward a second end 104 of the instrument 100 and the drill engagement portion 130 extends from the second end 104 toward the first end 102 of the instrument 100. The alignment assembly 300 may be coupled and decoupled from the drill engagement portion 130 as well as translated along the drill engagement portion 130. The drill engagement portion 130 engages the driving member 430. The drill engagement portion 130 couples with a drill for rotating the drill bit 478. The alignment assembly 300 allows for positioning and translating the driving member 430 relative to the tibial cutting guide 110. The driving member 430 allows for drilling of openings through the tibial cutting guide 110 for receiving a tibial implant (not shown).

With continued reference to FIGS. 1-20, the coupling portion or drill engagement portion 130 is shown. The coupling portion 130 has a first end 132 and a second end 134. The first end 132 of the coupling portion 130 couples to the driving member 430. The second end 134 of the coupling portion 130 couples to a drill (not shown). The coupling portion 130 includes a drill coupling member 140, a drive housing 160 positioned adjacent to and coupled to the coupling member 140, and a translation block 180 that couples to the drive housing 160. The coupling portion 130 also includes a first drive member 230 and a second drive member 250. The drive members 230, 250 are received within the drill coupling member 140, the drive housing 160, and the translation block 180.

The drill coupling member 140, as shown in FIGS. 1-20, includes a head portion 142 and a shaft 146 extending from the head portion 142. The head portion 142 may include recessed regions 154 inset into the head portion 142. The recessed regions 154 may be, for example, two recessed regions 154 positioned on opposite sides of the head portion 142. The head portion 142 includes an opening 144 extending into the head portion 142. At least a portion of the opening 144 may be, for example, threaded. The opening 144 may receive a portion of the first drive member 230. The first drive member 230 may have a threaded end 234 for engaging the opening 144 of the head portion 142. The shaft 146 may include a first portion 148 and a second portion 150 separated by the groove 152. The first portion 148 may be, for example, tapered along the length. The second end coupled to the groove 152 may have a smaller diameter than the first end coupled to the head portion 142. The smallest diameter of the first portion 148 may have, for example, the same diameter as the second portion 150. The second end may have the recessed regions 154 of the head portion 142 positioned adjacent to the first portion 148. The second portion 150 and the groove 152 of the drill coupling member 140 are sized and shaped or configured to engage the drill.

The drive housing 160 may include a body 162 and a tapered portion 164 extending and tapering away from the body 162, as shown in FIGS. 1-20. The body 162 may have planar portions and curved or rounded portions around the circumference of the body 162. The drive housing 160 may also include a through hole 166 extending from the first end to the second end of the drive housing 160 through the body 162 and tapered portion 164. The drive housing 160 may also include a first screw hole 168 and a second screw hole 170 extending through a portion of the body 162 to the first end of the drive housing 160. A portion of the screw holes 168, 170 may be, for example, recessed into a portion of the tapered portion 164. The first and second screw holes 168, 170 may be, for example, configured or sized and shaped to receive a first and second fastener 172, 174, respectively. The fasteners 172, 174 may be, for example, screws, bolts, or the like. The fasteners 172, 174 may extend through the screw holes 168, 170 to secure the drive housing 160 and the translation block 180.

FIGS. 1-20 also show the block or translation block 180 with a housing 182 and a base 200 coupled to and extending from the housing 182. The housing 182 may have, for example, a first recessed region 184 extending into the block 180 from a second end into the housing 182. The housing 182 may also include a second recessed region 186 inset into the housing 182 from the first recessed region 184 toward the first end of the block 180. The second recessed region 186 may have, for example, a circumference smaller than the circumference of the first recessed region 184. The housing 182 may further include a third recessed region 188 inset into the housing 182 from the second recessed region 186 toward the first end of the block 180. The third recessed region 188 may have, for example, a circumference smaller than the circumference of the second recessed region 186. Thus, the circumference of the first recessed region 184 is larger than the third recessed region 188. The block 180 also includes a through hole 190 extending through the housing 182 and the base 200 from the first end of the block 180 to the second end. The through hole 190 may be positioned near a first side of the block 180. The through hole 190 may, for example, overlap with a portion of the first recessed region 184. The through hole 190 may also be, for example, positioned adjacent to and spaced apart from the second and third recessed regions 186, 188. The housing 182 further include a first screw hole 192 and a second screw hole 194. The screw holes 192, 194 may be positioned on opposite sides of the through hole 190. The screw holes 192, 194 may be, for example, positioned to engage the fasteners 172, 174 after the fasteners 172, 174 pass through the drive housing 160. The screw holes 192, 194 may align with the first and second screw holes 168, 170 of the drive housing 160 when the translation block 180 and drive housing 160 are assembled.

The base 200 may include a first portion 202 and a foot 204 extending away from a first end of the block 180. The housing 182 of the block 180 is coupled to the second end of the base 200. The foot 204 extends away from a first side of the first portion 202 and a portion of the housing 182 extends away from a second side of the first portion 202. The first side is opposite the second side. The housing 182 may be, for example, wider than the width of the first portion 202. The foot 204 may have a through hole 206 extending through the foot 204 from a first end to a second end. The through hole 206 may be, for example, threaded along at least a portion of the hole 206 extending between the first end and the second end. The base 200 may also include a channel 208 inset into a first side of the first portion 202. The channel 208 may extend from the first end of the block 180 toward the housing 182 at the second end of the block 180. The first portion 202 also includes a cutout 210 extending into the first portion 202 from a top surface of the block 180. The cutout 210 extends from the top surface into the channel 208. As shown in FIG. 17, the cutout 210 may have, for example, an oval or rectangular shape. The cutout 210 may also have a portion that is open to a first side of the first portion 202. The base 200 may also include a rail 212 extending from a second side of the first portion 202. The rail 212 may extend from the foot 204 toward the housing 182 at the second end of the block 180. The portion of the rail 212 near the housing 182 may have a ramp or tapered end 214. The rail 212 may also include a first groove 216 inset into the rail 212 from a top surface and a second groove 218 inset into the rail 212 from a bottom surface.

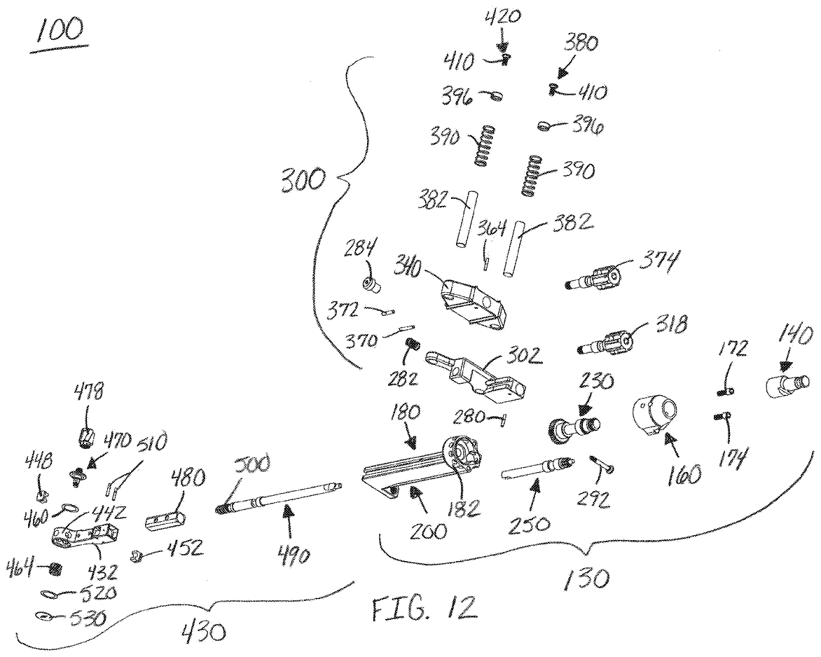

As shown in FIGS. 11-20, the first drive member 230 includes a shaft 232 with a first gear 240 at a first end. The shaft 232 includes a first protrusion 234 extending from the shaft 232 at the second end. At least a portion of the first protrusion 234 may be, for example, threaded. The shaft 232 includes a second protrusion 236 extending from the shaft 232 between the first protrusion 234 and the first gear 240. The second protrusion 236 may be, for example, separated from the first protrusion 234 by a lip 246. The first protrusion 234 has a first diameter, the lip 246 has a second diameter, and the second protrusion 236 has a third diameter. The third diameter is larger than the first and second diameters. The second diameter is smaller than the third diameter and the second diameter is larger than the first diameter. In addition, the shaft 232 includes a third protrusion 238 extending from the shaft 232 near or adjacent to the first gear 240. The third protrusion 238 is positioned between the second protrusion 236 and the first gear 240. The third protrusion 238 may have a fourth diameter and the fourth diameter may be, for example, the same or about the same size as the third diameter. The shaft 232 may include a fourth protrusion 242 positioned near the first end of the shaft 232. The fourth protrusion 242 may be positioned near or adjacent to the first gear 240. The fourth protrusion 242 is positioned between the first fear 240 and the first end of the shaft 232. The fourth protrusion 242 may have a fifth diameter and the fifth diameter may be, for example, the same or approximately the same size as the third and fourth diameters. The first gear 240 is positioned near the first end of the shaft 232. The first gear 240 may have, for example, a circumference and/or diameter larger than the circumference and/or diameter of each of the protrusions 234, 236, 238, 242. The first gear 240 may be positioned between the third protrusion 238 and the fourth protrusion 242. The drive member 230 may also have a head portion 244 positioned at the first end of the drive member 230. The head portion 244 may have a diameter smaller than the diameter of the shaft 232, protrusions 234, 236, 238, 242, and the first gear 240. The drive member 230 may be secured to the drill coupling member 140 by engaging the threads of the first protrusion 234 with the threaded opening 144 of the drill coupling member 140.

With continued reference to FIGS. 11-20, the second drive member 250 is shown. The second drive member 250 includes shaft portions 252, 258, 262, protrusions 256, 260, and a second gear 264. A first protrusion 256 is positioned between and extends from a first shaft portion 252 and a second shaft portion 258. A second protrusion 260 is positioned between and extends from the second shaft portion 258 and a third shaft portion 262. The first protrusion 256 is spaced apart from the second protrusion 260. The second gear 264 may be positioned between the third shaft portion 262 and a head portion 266. The second gear 264 may have, for example, a diameter that is the same size or smaller than the protrusions 256, 260. The second gear 264 is configured or sized and shaped to engage the first gear 240 within the coupled drive housing 160 and block 180. The head portion 266 may have a diameter smaller than the diameters of the shaft portions 252, 258, 262, the protrusions 256, 260, and the second gear 264. The second drive member 250 may also include a channel 254 extending into the first shaft portion 252 from the first end. The second drive member 250 may further include an opening 268 extending into the first shaft portion 252 from the first end toward the second end of the second drive member 250. The channel 254 may, for example, bisect the opening 268. The channel 254 may, for example, extend through the second drive member 250 from a first side to a second side.

The alignment assembly 300, as shown in FIGS. 1-12 and 21-28, includes a first or bottom alignment member 302, a second or top alignment member 340, a first coupling system 380, and a second coupling system 420. The first and second coupling systems 380, 420 may movably couple the first alignment member 302 to the second alignment member 340. The first alignment member 302 includes a base 304 and an extension member 310 coupled to and extending away from a top surface of the base 304 at a first end. The extension member 310 extends parallel with the base 304. The base 304 includes a first through hole 306 extending through the base 304 from a first end to a second end. The first through hole 306 may be, for example, positioned near a second side of the base 304. The base 304 may also include a channel 308 inset into the first side of the base 304. The channel 308 extends from a first end to a second end of the base 304. The base 304 also includes a second through hole 309 extending through the base 304 from a top surface to a bottom surface. The second through hole 309 may, for example, engage at least a portion of the first through hole 306. The second through hole 309 may also be positioned near a second end of the first alignment member 302. The extension member 310 is an elongated member with curved or rounded ends. The extension member 310 includes a first through hole 312 at a first or right side. The extension member 310 may also include an arm 313 extending from a second or left side of the extension member 310. The arm 313 includes a second through hole 314 at a second or left side. The through holes 312, 314 extend through the extension member 310 from a first or top surface to a second or bottom surface. The alignment assembly 300 may also include a protrusion 315 extending away from a bottom surface of the extension member 310. The protrusion 315 is coupled to and extending from the second side of the extension member 310. The protrusion 315 is positioned adjacent to the arm 313. The protrusion 315 may include a recess 316 extending into the protrusion 315 from a second side of the extension member 310. The protrusion 315 may also include a through hole 317 extending through the protrusion 315 from a first side to a second side through a portion of the recess 316.

The first alignment member 302 may also include a spring 282, a first fastener 284, and a second fastener 292 for coupling the first alignment member 302 of the alignment assembly 300 to the block 180 of the coupling portion 130. The first fastener 284 may include a head 286 and a shaft 288 extending away from the head 286. The shaft 288 may include an opening 290 extending into a second end of the shaft 288. The opening 290 may include, for example, threads. The spring 282 may be configured or sized and shaped to be positioned circumferentially around the shaft 288 of the first fastener 284. The second fastener 292 may include a head 294 and a shaft 296 extending away from the head 294. The shaft 296 may include a threaded end 298. The threaded end 298 may be received within and engage the opening 290 of the first fastener 284. The shaft 288 of the first fastener 284 is received within the recess 316. A portion of the shaft 296 of the second fastener 292 extends through the through hole 317 before engaging the shaft 288 of the first fastener 284. The head 294 of the second fastener 292 may be positioned outside the protrusion 315 for insertion through the cutout 210 for engaging the channel 208 of the base 200. The head 294 of the second fastener 292 and the channel 308 of the first alignment member 302 engage the channel 208 and rail 212 of the base 200, respectively, to translate the alignment assembly 300 along the coupling portion 130.

With continued reference to FIGS. 1-12 and 21-28, the alignment assembly 300 also includes a first securement knob 318. The first securement knob 318 includes a head 320 and a shaft 326 extending away from the head 320. The head 320 includes a drive opening 322 extending into the head 320 from the first end. The head 320 also includes through holes 324 near a second end of the knob 318 and extending through the knob 318 distal to the opening 322. The shaft 326 includes a first or central portion 328 extending from a second end of the knob 318. The shaft 326 also includes a protrusion 334 extending away from the shaft 326 between the first portion 328 and the head 320. The shaft 326 also includes a recess 330 inset into the protrusion 334 of the shaft 326 near a first end of the shaft 326. The recess 330 creates two portions of the protrusion 334. The shaft 326 further includes a threaded end 332 at a second end of the shaft 326. The central portion 328 of the shaft 326 is positioned between the protrusion 334 and the threaded end 332. The second through hole 309 may receive a pin 280 for engaging and securing the first securement knob or knob 318 within the base 304, while allowing for rotation of the knob 318. The pin 280 may engage, for example, the recess 330 of the first securement knob 318.

As shown in FIGS. 1-12 and 21-28, the second alignment member 340 includes a top surface 342 and a bottom surface 344. The second alignment member 340 also includes a first side member 346 and a second side member 348. The first side member 346 extends away from a first side of the second alignment member 340 and the second side member 348 extends away from a second side of the second alignment member 340. The side members 346, 348 may have, for example, a triangular shape. The second alignment member 340 may also include a first through hole 350 and a second through hole 352. The first through hole 350 is positioned in and extends through a point of the first side member 346. The second through hole 352 is positioned in and extends through a point of the second side member 348. The through holes 350, 352 extend through the second alignment member 340 from the top surface 342 to the bottom surface 344. The second alignment member 340 also includes at least one first protrusion 354 extending away from the top surface 342 of the alignment member 340 and at least one second protrusion 368 extending away from the bottom surface 344. The protrusions 354, 368 may extend between the first end and the second end of the alignment member 340. The at least one first protrusion 354 may be, for example, two protrusions 354 with a first at least one first protrusion 354 adjacent to the first side member 346 and a second at least one first protrusion 354 adjacent to the second side member 348. The at least one first protrusion 354 may have, for example, a triangular shape. The at least one second protrusion 368 may be, for example, two protrusions 368 with a first at least one second protrusion 368 adjacent to the first side member 346 and a second at least one second protrusion 368 adjacent to the second side member 348. The at least one second protrusion 368 may have, for example, a triangular shape. The at least one first protrusions 354 on the top surface 342 may be aligned with and/or as a mirror image of the at least one second protrusions 368 on the bottom surface 344.

The second alignment member 340 also includes a securement opening 356 extending from the first end to the second end of the second alignment member 340. The securement opening 356 is configured to receive a second securement knob 374. The second securement knob 374 may be, for example, the same or similar to the first securement knob 318 and which will not be described in detail here for brevity's sake. The second alignment member 340 also includes a first alignment pin opening 358 and a second alignment pin opening 360. The alignment pin openings 358, 360 extend into a portion of the second alignment member 340 from a first end. The first and second pin openings 358, 360 are configured or sized and shaped to receive first and second alignment pins 370, 372, respectively. The first alignment pin opening 358 is positioned on a first side of the securement opening 356. The second alignment pin opening 360 is positioned on a second side of the securement opening 356. The second alignment member 340 may also include a securement pin opening 362 extending through the alignment member 340 from the top surface 342 to the bottom surface 344 and into at least a portion of the securement opening 356. The securement pin opening 362 receives a securement pin 364 that engages the recess 330 in the second securement knob 374.

The first alignment member 302 is coupled to the second alignment member 340 by the first and second coupling systems 380, 420. As shown in FIGS. 1-12 and 21-28, the first coupling system 380 includes a rod 382, a spring 390, a retaining member 396, and a screw 410. The rod 382 includes a first end 384 and a second end 388. The first end 384 includes an opening or threaded opening 386 for receiving a portion of the screw 410. The second end 388 of the rod 382 is received within the through hole 312 of the first alignment member 302. The spring 390 is positioned or received circumferentially around the rod 382. The spring 390 includes a first end 392 and a second end 394. The first end 392 of the spring 390 engages the second alignment member 340 and the second end 394 of the spring 390 engages the first alignment member 302. The retaining member 396 includes a first end or top 398 and a second end or bottom 400. The retaining member 396 also includes a first recess 402 extending into the first end 398 of the retaining member 396 and a second recess 404 extending into the second end 400 of the retaining member 396. The recess 404 is, for example, configured or sized and shaped to receive the first end 384 of the rod 382. The recess 402 is, for example, configured or sized and shaped to receive a head 412 of the screw 410. The retaining member 396 also includes a through hole 406 extending through the retaining member 396 from a first end to a second end. The through hole 406 extends, for example, through a central axis or center point of the retaining member 396.

The screw 410 includes a head 414 and a shaft 416 extending from the head 414. The head 414 includes threads along at least portion of the shaft 416. The head 414 may be received within the first recess 402 of the retaining member 396. The shaft 416 may extend through the through hole 406 of the retaining member 396. The shaft 416 may be received within and coupled to the threaded opening 386 of the rod 382. The head 414 may also include a drive opening 414 extending into the first end of the head. The drive opening 414 may be used for rotating the screw 410. The screw 410 is inserted through the through hole 406 of the retaining member 396 and into the threaded opening 386 of the rod 382 to secure the first coupling system 380 between the alignment members 302, 340. The first coupling system 380 extends through and engages the first through hole 312 of the first alignment member 302 and the first through hole 350 of the second alignment member 340. The second coupling system 420 may be, for example, the same or similar to the first coupling system 380 and which will not be described again here for brevity's sake. The second coupling system 420 extends through and engages the second through hole 314 of the first alignment member 302 and the second through hole 352 of the second alignment member 340. Once the coupling systems 380, 420 are inserted between the alignment members 302, 340, the alignment members 302, 340 may, for example, move relative to each other along the rods 382.

With continued reference to FIGS. 1-12 and 29-36, the driving member 430 is shown. The driving member 430 may include a housing 432, a sleeve 480, and a drive shaft 490. The housing 432 may include a through hole 434 extending into the housing 432 from the second end toward the first end. The housing 432 may also include a first cavity 436 extending from a first side of the housing 432 into the through hole 434. The housing 432 may further include a second cavity 438 extending from a second side of the housing 432 into the through hole 434. The housing 432 may also include securement pin holes 440 extending through the housing 432 from the top surface to the bottom surface. The holes 440 may extend from the exterior of the housing into the cavities 436, 438. In addition, the housing 432 may have a protrusion 442 extending from a first side of the housing 432 near a first end. The first end of the housing 432 may have, for example, one or more rounded, curved or radiused edges, for example, in one embodiment the protrusion 442 may be rounded, curved or radiused. An opening 444 extends into the housing 432 from a top surface toward the bottom surface. At least a portion of the opening 444 extends into the protrusion. The opening 444 may also include a first lip 446 inset into a top surface of the housing 432 and surrounding the opening 444. The opening 444 may further include a second lip 447 inset into a bottom surface of the housing 432 and surrounding the opening 444. The driving member 430 may also include a first sidewall 448 for insertion into the first cavity 436 and a second sidewall 452 for insertion into the second cavity 438. The first sidewall 448 may include a first recessed region 450 on the interior surface of the first sidewall 448 to surround the through hole 434 on a first side. The first sidewall 448 may also include a first securement pin hole 456 extending through the first sidewall 448 from a top surface to a bottom surface. The second sidewall 452 may include a second recessed region 454 on the interior surface of the second sidewall 452 to surround the through hole 434 on a second side. The second sidewall 452 may also include a second securement pin hole 458 extending through the second sidewall 452 from a top surface to a bottom surface. The sidewalls 448, 452 may be secured to the housing 432 with pins 510 inserted through holes 440 in the housing 432 and openings 456, 458 in the sidewalls 448, 452, respectively.

The driving member 430 may further include a first washer 460, a first worm gear 464, a rotating member 470, a drill bit 478, a second washer 520, and an end cap 530. The first washer 460 is received within the opening 444 and positioned on top of the first lip 446. The first washer 460 may include a through hole 462 extending through the first washer 460 from the top surface to the bottom surface. The top surface of the first washer 460 may be positioned adjacent to a bottom surface of the base 472 of the rotating member 470. The first worm gear 464 is positioned within the opening 444. The first worm gear 464 may include a recessed region 466 extending into the worm gear 464 from a top surface toward a bottom surface. The first worm gear 464 may also include a through hole 468 extending through the worm gear 464 from the top surface to the bottom surface. The through hole 468 may have, for example, a rectangular or other polygonal shape. The rotating member 470 may include a base 472, a first shaft 474 extending away from a first or top surface of the base 472, and a second shaft 476 extending away from a second or bottom surface of the base 472. The first shaft 474 may have, for example, a round or circular shape. The first shaft 474 may have, for example, a threaded portion 475 along at least a portion of the first shaft 474. The second shaft 476 may have, for example, a square or polygonal shape to correspond to the shape of the through hole 468 of the first worm gear 460. The rotating member 470 may also include a protrusion 477 extending from the second shaft 476 adjacent to the base 472. The protrusion 477 is received within and engages recessed region 466 of the first worm gear 464. Further, the driving member 430 includes a drill bit 478 with an opening 479 extending into the drill bit 478 from a second or bottom surface toward the first or top surface. The opening 479 is configured or sized and shaped to receive the first shaft 474 of the rotating member 470. The second washer 520 is received within the opening 444 and positioned below the second lip 447. The second washer 520 may include a through hole 522 extending through the second washer 520 from the top surface to the bottom surface. The end cap 530 includes a base 532 with a protrusion 534 extending away from a top surface of the base 532. The second washer 520 may be positioned to surround the protrusion 534. The second washer 520 also includes a through hole 536 extending through the second washer 520. The through hole 536 extends through the base 532 and the protrusion 534. The through hole 536 may be, for example, threaded to receive the threaded portion 475 of the rotating member 470.

The driving member 430 may also include a sleeve 480 that couples to the second end of the housing 432. The sleeve 480 may have a first end 482 and a second end 484. The sleeve 480 may also include a channel 486 extending through the sleeve 480 from the first end 482 to the second end 484. The channel 486 is configured or sized and shaped to receive a portion of the drive shaft 490. The drive shaft 490 includes a shaft portion 492 and a second worm gear 500. The drive shaft 490 may also include a first protrusion 494 extending from the shaft portion 492 near a midpoint of the shaft portion 492. The shaft portion 492 also includes a second protrusion 496 extending from the shaft portion 492 between the second worm gear 500 and the first protrusion 494. The shaft portion 492 may further include a third protrusion 497 extending from the shaft portion 492 between the second protrusion 496 and the second worm gear 500. The third protrusion 497 has a diameter larger than the diameter of the first and second protrusions 494, 496. The drive shaft 490 also includes a first recessed region 498 and a second recessed region 499. The first recessed region 498 is positioned between the second worm gear 500 and the third protrusion 497. The second recessed region 499 is positioned between the first protrusion 494 and the second protrusion 496. The diameter of the recessed shaft regions 498, 499 may be, for example, smaller than the diameter of the shaft portion 492, protrusions 494, 496, 497, and second worm gear 500. The second worm gear 500 may be, for example, positioned near the first end of the drive shaft 490. The drive shaft 490 may also include an alignment tab 502 at the second end of the drive shaft 490. In addition, the alignment pin 504 extends from the alignment tab 502 to the second end. The alignment tab 502 may be for example, flat or planar, while the alignment pin 504 is round or cylindrical.

A surgical method for using the instrument 100 may include obtaining an instrument 100 and a tibial cutting guide 110 after a tibial resection cut has been performed as part of a total ankle resection (TAR) procedure. The tray 112 may be positioned such that the top surface of the tray 112 abuts the resected surface of the distal tibia. Next, pins (not shown) may be inserted through openings 128 in the tibial coupling element 122 to couple the tibial cutting guide 110 with an anterior portion of the tibia. In addition, the compression screw 108 may extend through the securement opening 126 of the tibial cutting guide 110 to assist with securely placing the tibial cutting guide 110 to the tibia. The instrument 100 may also be coupled to the tibial cutting guide 110 prior to or after the tibial cutting guide 110 is secured to the tibia. The tibial cutting guide 110 can be attached to the alignment assembly 300 before the drill bit 478 is positioned relative to the tibia. More preferably, the tibial cutting guide 110 may be coupled to the tibia with k-wires (not shown) before the instrument 100 is positioned with the drill bit 478 under the resected surface of the tibia. In addition, the instrument 100 may be inserted into position relative to the distal tibia before the talus cut is performed. After the drill bit 478 is positioned under the tibia, the alignment assembly 300 may be translated along the base 200 to couple to the tibial cutting guide 110. When the instrument 100 is secured to the cutting guide 110, the drill bit 478 is positioned between the first arm 114 and the second arm 116 within the drill channel 117. To ensure that the drill bit 478 is correctly positioned within the drill channel 117, fluoroscopy may then be performed intraoperatively from a medial or lateral shot. The fluoroscopy may allow the surgeon to visualize the position of the drill bit 478 adjacent the resected surface of the distal tibia, and/or at least a portion of the housing 432 relative to the cutouts or windows 119 recessed within the top surface 118 of the tibial tray 112 and/or the recess 121 extending to the bottom surface 120 of the tibial tray 112. If the drill bit 478 is not positioned correctly, the surgeon may move its position and then do additional fluoroscopy until the desired position is achieved. Once the drill bit 478 is positioned correctly, then the procedure may continue. The shaft 146 of the coupling member 140 may be coupled with a powered handle or drill before or after fluoroscopy is performed. Next, the surgeon may drill into the distal tibia. As force is applied to the drill 100, the housing 432 may fit within the recess 121 of the tibial tray 112 to allow for determination of whether the drill bit 478 is aligned and able to be fully seated in the distal tibia or if the drill bit 478 is misaligned. Further, as force is applied to the drill 100, at least a portion of the housing 432 may occlude at least a portion of the windows 119 when viewed from a medial or lateral perspective. Once the windows 119 are completely occluded by the housing 432, the drill bit 478 has been bottomed out in the distal tibia (e.g., drilled as far proximally as possible). The housing 432 must be aligned with the recess 121 of the tibial tray 112 in order for the drill bit 478 to drill to the desired depth in the resected surface of the distal tibia. After the desired opening is drilled into the tibia, the instrument 100 and tibial cutting guide 110 may be decoupled from each other and the tibia. Finally, the TAR procedure may proceed.

The drilling into the distal tibia may be done by applying an upward force to at least part of at least one of the coupling portion 130, alignment assembly 300, and driving member 430 such that the tip of the drill bit 478 contacts the resected surface of the distal tibia. The force may be applied by the surgeon or using an additional instrument, for example, a distractor that engages the housing 432. Alternatively, the force may be applied by applying force to the first alignment member 302 and the second alignment member 340 to translate the housing 432 and thus, move the drill bit 478 into the resected surface of the distal tibia. Once the drill bit 478 contacts the resected surface, the powered handle or drill (not shown) may be actuated to rotate the first drive member 230, which in turn rotates the second drive member 250, the drive shaft 490, and the first worm gear 464 to initiate rotation of the drill bit 478. As the drill bit 478 is rotated and force is applied, the drill bit 478 may pass through the drill channel 117 of the tibial tray 112 to drill the opening in the tibia for receiving a tibial implant.

In another embodiment, drilling into the distal tibia may include inserting a first smaller drill bit 478 into contact with the resected surface and drilling a first opening into the distal tibia. The drill 100 may then be removed and the first drill bit 478 replaced (e.g., after drilling with the first drill bit 478 was known to be completed based on complete occlusion of the windows 119 by the housing 432) with a larger drill bit 478 or a drill 100 with a larger drill bit 478 may be obtained. The tibial trial 112 may be removed through, for example, the drill channel feature or dovetail to allow additional space for the following step. Then, the drill 100 with the larger drill bit 478 may be inserted into contact with the resected surface. The tibial trial may then be replaced such that the drill channel 117 receives the larger drill bit 478 therein. A second opening may be drilled over the first opening in the distal tibia and/or the first opening may be extended further distally in the tibia. The second or larger drill bit 478 may have, for example, a taller height than the first drill bit 478. The larger drill bit 478 may also have, for example, the same diameter as the first drill bit 478. Alternatively, the larger drill bit 478 may have, for example, the same diameter as the first drill bit 478 allowing for insertion of the larger drill bit 478 into the first opening to ensure proper alignment. Once the larger drill bit 478 is positioned on the resected surface of the distal tibia if the diameter of the larger drill bit 478 is the same as the first drill bit 478, then the larger drill bit 478 may be inserted into the first opening drilled with the first drill bit 478. The tibial trial 112 may then be replaced using the same drill channel feature. Then, the surgeon may drill into the distal tibia a second time using the larger drill bit 478, where the housing 432 may occupy at least a portion of the windows 119 to indicate that the larger drill bit 478 has bottomed out in the distal tibia. If the desired opening is created with the larger drill bit 478, then the instrument 100 and the tibial cutting guide 110 may be removed from the patient and the procedure completed. If the desired opening is not created, then a third drill bit 478 could be used to create the desired opening for the stem of the tibial implant.

It is also contemplated that in addition to different size drill bits 478 for drilling the desired opening in the distal tibia, that different size tibial trials 112 may also be used. The different size tibial trials 112 may have, for example, thinner or thicker heights between the top surface 118 and the bottom surface 120. The thickness of the tibial trials 112 may allow for use of thinner tibial trials 112 with larger drill bits 478 to enable use of the drill 100 prior to performing any talus resection. Further, it is also contemplated that the drill bit 478 could have a first height in a first or seated position and a second height in a second or actuated position. The drill bit 478 would be translatable from the first position to the second position after insertion into position adjacent to the resected surface of the distal tibia.

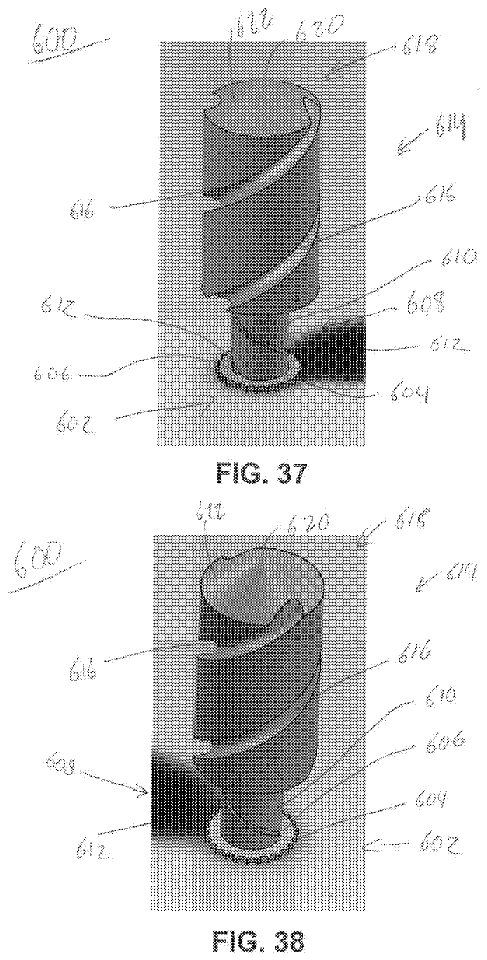

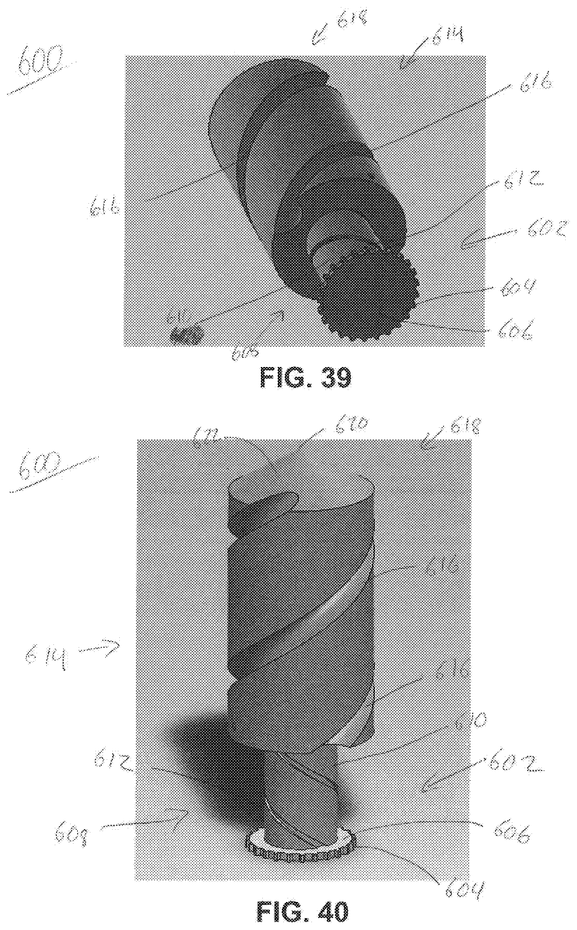

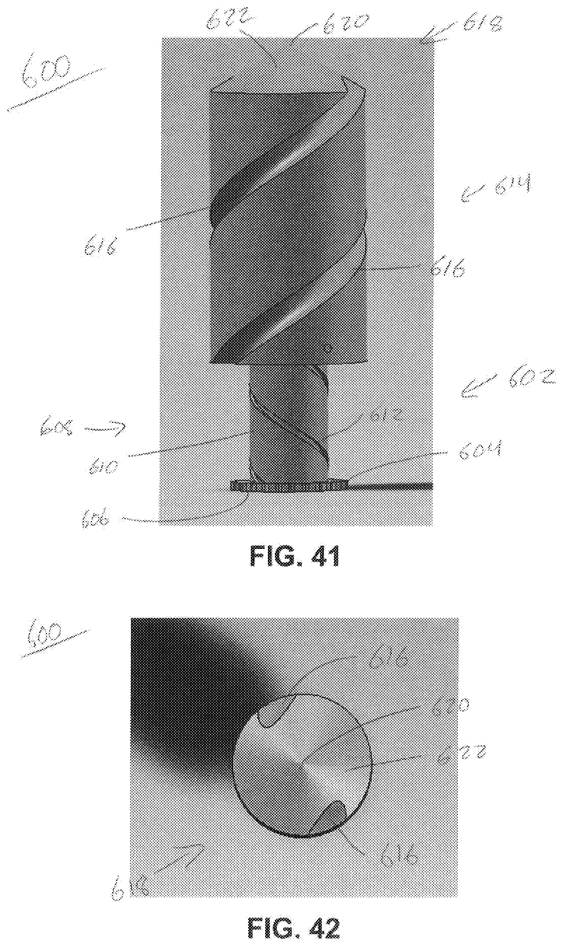

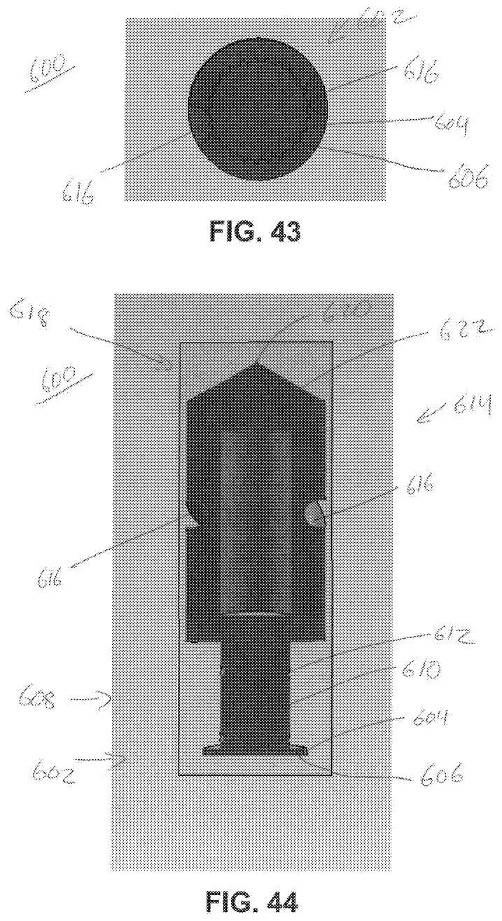

Referring now to FIGS. 37-44, a drill bit 600 is shown, according to an exemplary embodiment. The drill bit 600 may be implemented in conjunction with one or more of the instruments and/or alignment systems shown and described previously herein, for example the instrument 100 and/or the alignment assembly 300. Further, the drill bit 600 may be implemented in place of one or more components of an instrument or alignment assembly, for example the drill bit 478 of the instrument 100. In some embodiments, the drill bit 600 may be releasably couplable with one or more components of the instrument 100, for example the first shaft 474 of the rotating member 470, where the rotating member 470 is engaged and/or coupled with other components of the instrument 100. In some aspects, the drill bit 600 may be otherwise engaged and/or coupled with the instrument 100 and components thereof according to geometric features of the drill bit 600 shown in FIGS. 37-44 and described subsequently herein.

The drill bit 600 is shown to include a base portion 602, which is positioned opposite a shaft 608 from a body portion 614. The base portion 602 is shown to have a substantially circular geometry (e.g., a cylindrical volume with a height lesser than that of the shaft 608) with a plurality of teeth 604 disposed about the circumference/outer surface of the base portion 602. In some aspects, the teeth 604 may be sized, positioned, and/or otherwise configured to engage with one or more components of the instrument 100, for example the first and/or second worm gears 464 and 500, respectively. The base portion 602 is shown to include a top surface 606, which is positioned adjacent to the shaft 608 as the shaft 608 has a lesser lateral dimension (e.g., radius, diameter, circumference) than that of the base portion 602. As shown, the base portion 602 is integral with the shaft 608, but in some portions may be modular and releasably couplable with the shaft 608 via a threading or other coupling means.

The shaft 608 is shown to include grooves 612 disposed within an outer surface 610 of the shaft 608, according to an exemplary embodiment. As shown, the grooves 612 extend from the top surface 606 of the base portion 602, and along the height of the shaft 608 until the shaft 608 abuts the body portion 614 (which, as shown, the shaft 608 is integral with). The grooves 612, as shown in at least FIG. 37, include multiple grooves 612 disposed substantially diametrically opposite the shaft 608 from one another and having a curved path between the top surface 606 of the base portion 602 and the top of the shaft 608. Accordingly, the grooves 612 may be configured to receive one or more protrusions of the instrument 100 (and/or a component thereof) at least partially wherein such that rotation of the drill bit 600 while the protrusion(s) is(are) disposed within the grooves 612 raises or lowers the drill bit 600 relative to the protrusion(s) and/or other adjacent components of the instrument 100. In some embodiments, the shaft 608 may include various numbers of grooves 612 having various sizes and/or paths between the top surface 606 of the bottom portion 602 and the top of the shaft 608. For example, the grooves 612 may include one, two, three, four, or any other number of grooves, one or more of which may include curved paths extending at least partially around the outer surface of the shaft 608. Further, said grooves 612 may have various degrees of inclination, which accordingly correspond to how much rotation of the drill bit 600 is required to raise/lower the drill bit 600 a set amount (where a lesser angle of inclination results in a longer groove between the top surface 606 of the base portion 602 and the top of the shaft 608 and thus requires a greater amount of rotation of the drill bit for the protrusion(s) to move within the grooves 612 from adjacent the top surface 606 of the base portion 602 to the opposite point of the grooves 612 at the top of the shaft 608).

The body portion 614, which is shown to be integral with the shaft 608 (but in some embodiments may be modular and releasably couplable with one another via a threading or other coupling mechanism) is shown to have a substantially cylindrical geometry with a greater lateral dimension than that of the shaft 608 and the base portion 602. Further, the body portion 614 is shown to have a greater height than that of the base portion 602 and the shaft 608, as shown in at least FIG. 40. As shown, the body portion 614 is integral with the shaft 608. However, in some aspects, the body portion 614 may be modular and releasably couplable with one or both of the shaft 608 and/or the base portion 602.

The body portion 614 is shown to include a plurality of cutting flutes 616, shown in at least FIG. 40 as a pair of cutting flutes extending at least partially around the outer surface of the body portion 614. In some aspects, the cutting flutes 616 may include a single cutting flute, or may include three or more cutting flutes. As shown, the cutting flutes 616 extend from a bottom edge (e.g., adjacent the shaft 608) of the outer surface of the body portion 614 to a top edge (e.g., opposite the body portion 614 from the bottom edge) of the body portion 614. The cutting flutes 616 are shown to be positioned such that at least a portion of each cutting flute 616 may be substantially opposite the body portion 614 from a corresponding portion of a different cutting flute 616 (e.g., diametrically opposite). The cutting flutes 616 may form a substantially consistent angle (e.g., an inclination angle, which as shown may be substantially acute) relative to the bottom edge of the body portion 614 as the cutting flutes extend toward the top edge of the body portion 614. In some embodiments, for example that shown in FIG. 40, one or more of the cutting flutes 616 may terminate beyond the top edge of the body portion 614, for example on a top surface 622 of a top portion 618 of the drill bit 600. In some aspects, one or more of the cutting flutes 616 may terminate at a substantially rounded geometry. As shown in at least FIG. 44, the drill bit 600 may be at least partially hollow, with a closed volume positioned within the body portion 614 of the drill bit 600.

The top portion 618 is shown to have a substantially cone-shaped geometry, with a tip 620 forming the apex of said cone-shaped geometry, according to an exemplary embodiment. As shown, the top portion 618 is integral with the body portion 614, although in some aspects the top portion 618 may be modular and releasably couple with the body portion 614. In some aspects, the cutting flutes 616 may extend from the body portion 614 onto at least a portion of the top surface 622, for example terminating at a point on the top surface 622 as shown in at least FIG. 40. In some aspects, one or more of the cutting flutes 616 may terminate at the top 620 of the top portion 618.

As may be recognized by those of ordinary skill in the art based on the teachings herein, numerous changes and modifications may be made to the above-described and other embodiments of the present disclosure without departing from the scope of the disclosure. The components of the implants as disclosed in the specification, including the accompanying abstract and drawings, may be replaced by alternative component(s) or feature(s), such as those disclosed in another embodiment, which serve the same, equivalent or similar purpose as known by those skilled in the art to achieve the same, equivalent or similar results by such alternative component(s) or feature(s) to provide a similar function for the intended purpose. In addition, the implants may include more or fewer components or features than the embodiments as described and illustrated herein. Accordingly, this detailed description of the currently-preferred embodiments is to be taken in an illustrative, as opposed to limiting of the disclosure.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The disclosure has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the disclosure be construed as including all such modifications and alterations.

What is claimed is:

1. An instrument, comprising:
a coupling portion;
an alignment assembly coupled to the coupling portion; and
a driving member coupled to the alignment assembly, wherein a drill bit is positioned perpendicular to a housing of the driving member.

2. The instrument of claim 1, wherein the alignment assembly translates along a translation block of the coupling portion.

3. The instrument of claim 2, wherein the coupling portion comprises:
a drill coupling member;
a drive housing positioned adjacent to the drill coupling member;
a translation block coupled to the drive housing by at least one fastener;
a first drive member positioned within at least a portion of the drive housing and extending through the drive housing into the drill coupling member; and
a second drive member positioned within at least a portion of the translation block and the drive housing.

4. The instrument of claim 3, wherein the first drive member rotatably engages a second gear of the second drive member.

5. The instrument of claim 3, wherein the translation block comprises:
a base;
a foot coupled to and extending from a first end of the base; and
a housing coupled to and extending from a portion of a second end of the base;
wherein the base comprises:
a channel inset into a first side of the base; and
a rail extending from a second side of the base, wherein the alignment assembly engages the channel and the rail to translate along a length of the coupling portion.

6. The instrument of claim 1, wherein the alignment assembly comprises:
a first alignment member;
a second alignment member; and
at least one coupling system movably connecting the first alignment member to the second alignment member.

7. The instrument of claim 6, wherein the at least one coupling system comprises:
a first coupling system positioned on a first side of the alignment assembly; and
a second coupling system positioned on a second side of the alignment assembly.

8. The instrument of claim 6, wherein the at least one coupling system comprises:
a rod with a first end and a second end;
a retaining member engaging a first end of the rod;
a spring moveably positioned around the rod; and
a screw extending through the retaining member and into a portion of the first end of the rod.

9. The instrument of claim 1, wherein the driving member comprises:
a housing;
a sleeve coupled to a second end of the housing;
a drive shaft extending through the sleeve and a portion of the housing;
a first worm gear positioned within the housing and engaging a portion of the drive shaft; and
a rotating member with a first shaft and a second shaft, wherein the first shaft of the rotating member engages a through hole of the first worm gear, and
wherein the drill bit engages the second shaft of the rotating member.

10. The instrument of claim 9, wherein the first worm gear engages a second worm gear of the drive shaft.

11. The instrument of claim 10, wherein a second end of the drive shaft engages a second drive member of the coupling portion.

12. The instrument of claim 11, wherein the first worm gear, the second worm gear, a first gear of the first drive member, and a second gear of the second drive member are rotatably coupled.

13. A surgical method for an arthroplasty procedure, comprising:
obtaining an instrument and a tibial cutting guide;
aligning a drill bit of the instrument with a tibial resection;
translating a coupling portion of the instrument into position adjacent to the tibial resection;
translating the drill bit to drill an opening in the tibia; and
removing the instrument after an opening is drilled.

14. The surgical method of claim 13, further comprising:
attaching the tibial cutting guide to the coupling portion;
securing to the tibial cutting guide to a tibia; and
removing the tibial cutting guide after an opening is drilled.

15. The surgical method of claim 14, wherein aligning a drill bit of the instrument with a tibial resection comprises:
aligning a drill bit of the instrument with at least one window in a tibial tray of the tibial cutting guide.

16. The surgical method of claim 14, further comprising:
coupling a shaft of a coupling member of the instrument to a powered handle.

17. The surgical method of claim 16, further comprising:
actuating the powered handle to rotate the drill bit and drill the tibia.

18. The surgical method of claim 13, wherein translating the drill bit to drill into the tibia comprises:
applying force to at least a portion of the instrument.

19. The surgical method of claim 13, wherein the instrument comprises:
a coupling portion;
an alignment assembly coupled to the coupling portion; and
a driving member coupled to the alignment assembly, wherein a drill bit is positioned perpendicular to a housing of the driving member.

20. A drilling system, comprising:
an instrument, comprising:
a coupling portion;
an alignment assembly coupled to the coupling portion; and
a driving member coupled to the alignment assembly, wherein a drill bit is positioned perpendicular to a housing of the driving member; and
a tibial cutting guide, wherein the instrument couples to the tibial cutting guide.
